(19) United States
(12) Patent Application Publication
(10) Pub. No.: US 2016/0038518 A1
(43) Pub. Date: Feb. 11, 2016

(54) METHODS FOR TREATING PSORIASIS AND VASCULAR INFLAMMATION

(71) Applicant: VASCULAR BIOGENICS LTD., Or Yehuda (IL)

(72) Inventors: Naamit SHER, Rechovot (IL); Andrea LEUBITZ, Efrat (IL); Eyal BREITBART, Hashmonaim (IL)

(21) Appl. No.: 14/379,058

(22) PCT Filed: Feb. 15, 2013
(86) PCT No.: PCT/IB2013/000780

§ 371 Date: Aug. 15, 2014

Related U.S. Application Data
(60) Provisional application No. 61/723,605, filed on Nov. 7, 2012.
(60) Provisional application No. 61/611,276, filed on Mar. 15, 2012.
(60) Provisional application No. 61/599,493, filed on Feb. 16, 2012.

(51) Int. Cl. A61K 31/685; A61K 47/02; A61K 45/06

(52) U.S. Cl. CPC A61K 31/685; A61K 45/06; A61K 47/02

(57) ABSTRACT

Described are methods of decreasing vascular inflammation in a subject suffering from a chronic autoimmune or chronic inflammatory disease by administering to the subject a therapeutically effective amount of VB-201, wherein the therapeutically effective amount is from about 20 mg/day to about 160 mg/day, optionally administered in two daily sub-doses. Also described are methods of treating inflammation associated with an implant (e.g., a breast implant) in a subject by administering to the subject a therapeutically effective amount of VB-201, wherein the therapeutically effective amount is from about 20 mg/day to about 160 mg/day, optionally administered in two daily sub-doses. Further described are methods of treating psoriasis (e.g., active plaque psoriasis) in a subject by administering to the subject a therapeutically effective amount of VB-201, wherein the therapeutically effective amount is from about 80 mg/day to about 160 mg/day, optionally administered in two daily sub-doses.

### BACKGROUND OF THE INVENTION

### Field of the Invention

The present invention relates to methods of decreasing vascular inflammation, e.g., in atherosclerosis, in a subject. The present invention further relates to methods of treating psoriasis, including moderate to severe psoriasis.

### BACKGROUND OF THE INVENTION

Oxidized phospholipids have previously been described as useful in the treatment of medical conditions such as, for example, cardiovascular diseases, cerebrovascular diseases and inflammatory diseases and disorders. International Patent Application No. PCT/IL2004/000453 (Publication No. WO04/106486) describes oxidized lipids for prevention and treatment of inflammation associated with endogenous oxidized lipids. An exemplary such compound is described and known as CI-201 [1-hexadecyl-2-(4′-carboxy)butyl-sn-glycero-3-phosphocholine]; also referred to as VB-201). VB-201 is an innate immune-modulator and member of a therapeutic class of oxidized phospholipid analogs termed lecinoxoids.

International Patent Application No. PCT/IL01/01080 (published as WO2002/041827) describes oxidized lipids for prevention and treatment of atherosclerosis and related diseases. International Patent Application Nos. PCT/IL01/01080 (published as WO2002/041827), PCT/IL2004/000453 (published as WO2004/106486) and PCT/IL2011/000010 (published as WO2011/083467) describe co-administration of an oxidized lipid with a statin, all of which are incorporated herein by reference in their entirety. PCT/IL2011/000010 (published as WO2011/083467) describes co-administration of VB-201 and atorvastatin to rabbits (Example 1 of WO2011/083467), as well as administration of VB-201 to patients having an elevated high sensitivity C-reactive protein level and who have been on a stable high dose of statin for at least 3 months (Example 3 of WO2011/083467), which is incorporated herein by reference in its entirety.

Statins, which are also known in the art as HMG-CoA reductase inhibitors, are a class of drugs used to lower cholesterol levels by inhibiting the enzyme HMG-CoA reductase, which plays an important role in the production of cholesterol. Statins are widely administered in order to treat cardiovascular disease, and in order to prevent development of cardiovascular disease in subjects exhibiting risk factors for cardiovascular disease, such as elevated cholesterol levels, diabetes, and/or high blood pressure. Statin administration reduces major coronary events by 27% to 37% versus placebo (see, e.g., Lancet 2002, 350:7-22; Lancet 1995, 333:1301-1307; Lancet 1994, 344:1383-1389; Lancet 2003, 361:1149-1157; Lancet 2004, 364:685-696).

Statins are associated with a number of adverse side effects, primarily elevated blood levels of liver enzymes and moderate muscle problems (e.g., myalgia, muscle cramps), but also gastrointestinal problems, polyneuropathy, and relatively severe muscle problems such as myositis, myopathy and rhabdomyolysis (which can lead to acute renal failure).

### BRIEF DESCRIPTION OF THE INVENTION

In some embodiments the present disclosure describes a method of treating vascular inflammation in a subject suffering from a chronic autoimmune or chronic inflammatory disease (e.g., psoriasis), the method comprising administering to the subject a therapeutically effective amount of VB-201.

In other embodiments, the present disclosure provides a method of decreasing vascular inflammation in a subject suffering from a chronic autoimmune or inflammatory disease (e.g., psoriasis), the method comprising administering to the subject a therapeutically effective amount of VB-201, wherein the vascular inflammation is measured using positron emission computed tomography (PET/CT) imaging. In some examples, the vascular inflammation, after administering the therapeutically effective amount to the subject for about 12 weeks, is reduced by at least about 6%, at least about 8%, at least about 10%, at least about 12%, or at least about 14% as compared to the vascular inflammation prior to the administering (base line).

In some embodiments, the present disclosure provides a method of treating vascular inflammation in a subject suffering from a chronic autoimmune or chronic inflammatory disease (e.g., psoriasis), the method comprising administering to the subject a therapeutically effective amount of VB-201, wherein the vascular inflammation is associated with a cardiovascular disease, a peripheral vascular disease, a coronary artery disease, a cerebral vascular disease, a renal artery stenosis, an ischemic disease, or an aortic aneurism. In preferred embodiments, the chronic autoimmune or chronic inflammatory disease is psoriasis.

In some embodiment, the vascular inflammation is associated with a cardiovascular disease, or disorder selected from the group consisting of occlusive diseases or disorders, atherosclerosis, a cardiac valvular disease, stenosis, restenosis, in-stent-stenosis, myocardial infarction, coronary arterial disease, acute coronary syndromes, congestive heart failure, angina pectoris, myocardial ischemia, thrombosis, Wegener's granulomatosis, Takayasu's arteritis, Kawasaki syndrome, anti-factor VIII autoimmune disease or disorder, necrotizing small vessel vasculitis, microscopic polyangiitis, Churg and Strauss syndrome, pauci-immune focal necrotizing glomerulonephritis, crescentic glomerulonephritis, antiphospholipid syndrome, antibody induced heart failure, thrombocytopenic purpura, autoimmune hemolytic anemia, cardiac autoimmunity, Chagas' disease or disorder, and anti-helper T lymphocyte autoimmunity. In some embodiment, the vascular inflammation is an inflammation of a carotid artery, an inflammation of an aorta, or both.

In some embodiments, the present disclosure provides a method of treating vascular inflammation in a subject suffering from a chronic autoimmune or chronic inflammatory disease (e.g., psoriasis), the method comprising administering to the subject a therapeutically effective amount of VB-201, wherein the vascular inflammation is associated with an ischemic heart disease, atherosclerosis, acute coronary syndrome, unstable angina, stable angina, angina pectoris or stroke. In preferred embodiments, the chronic autoimmune or chronic inflammatory disease is psoriasis.

In other embodiments, the present disclosure provides a method of treating or reducing inflammation associated with an implant in a subject in need thereof, the method comprising administering to the subject a therapeutically effective amount of VB-201. The term “implant” herein includes any human made device or artificial material that is implanted into a subject's (e.g., a human's) body. In some embodiments, the inflammation associated with an implant is reactive inflammation in response to the implants, which may affect tissues surrounding the implants. In some embodiments, the implant is a silicone, a saline, a metal, a plastic, or a polymeric implant. In some embodiments, the implant is a cosmetic implant, a prosthetic implant, a subdermal implant, a transdermal implant, a bone replacement implant, or a bone fracture repair device. In some embodiments, the implant is a drug delivery implant or a drug release implant. In some embodiments, the implant is an artificial joint, an artificial heart, an artificial heart valve, a testicular prosthesis, a breast implant, a dental implant, an ocular implant (e.g., artificial lens), a cochlear implant, a penile implant, a cardiac implant, a catheter, an implantable urinary continence device, a pacemaker, an electrode, a Hernia support device (e.g., nets), or a respirator tube. In some embodiments, the implant is a breast implant. In some embodiments the implant is a vascular implant.

In other embodiments, the present disclosure provides a method of treating severe psoriasis (psoriasis of category 4 according to the Physician. Global Assessment (PGA) scale), the method comprising administering to a subject in need thereof a therapeutically effective amount of VB-201, wherein the therapeutically effective amount is from about 20 mg/day to about 160 mg/day, from about 20 mg/day to about 80 mg/day, or from about 80 mg/day to about 160 mg/day, e.g., for a treatment period of at least about 8 weeks, at least about 12 weeks, at least about 16 weeks, or at least about 24 weeks.

In other embodiments, the present disclosure provides a method of treating moderate, severe, or worst psoriasis has ever been (psoriasis of categories 3-5 according to the Patient Global Assessment (PtGA) scale), the method comprising administering to a subject in need thereof a therapeutically effective amount of VB-201, wherein the therapeutically effective amount is from about 20 mg/day to about 160 mg/day, from about 20 mg/day to about 80 mg/day, or from about 80 mg/day to about 160 mg/day, e.g., for a treatment period of at least about 8 weeks, at least about 12 weeks, at least about 16 weeks, or at least about 24 weeks.

In some examples according to any of the embodiments described herein, the therapeutically effective amount is from about 20 mg/day to about 160 mg/day (e.g., from about 20 mg/day to about 80 mg/day). Other suitable ranges for the therapeutically effective amount are described herein. In other examples, the therapeutically effective amount is administered in two daily doses, e.g., about 12 hours apart from each other (Q12H). A two-times-per-day dosing regimen is particularly useful when the therapeutically effective amount is greater than about 80 mg/day.

Thus, in other embodiments, the present disclosure provides a method of treating psoriasis comprising administering to a subject in need thereof a therapeutically effective amount of VB-201, wherein the therapeutically effective amount is from about 80 mg/day to about 160 mg/day (e.g., about 160 mg/day) administered to the subject in 2 daily doses, e.g., about 12 hours apart from each other (Q12H). In one example, when the therapeutically effective amount of VB-201 is about 160 mg/day, the subject is administered about 80 mg in the morning and about 80 mg in the evening.

In other embodiments, the present disclosure provides a method of treating moderate to severe psoriasis in a subject in need thereof, the method comprising administering to the subject a therapeutically effective amount of VB-201 for a treatment period, wherein: a) the subject has a Psoriasis Area and Severity Index (PASI) score of 10 to 20 prior to the treatment period; b) the subject has a body surface area (BSA) of 10% to 30% prior to the treatment period; c) the subject was not treated with an anti-psoriatic biologic or an immunosuppressant drag prior to the treatment period; or any combination thereof.

In other embodiments, the present disclosure provides a method of treating psoriasis, the method comprising administering to a subject in need thereof a therapeutically effective amount of VB-201, wherein the therapeutically effective amount is from about 80 mg/day to about 160 mg/day (e.g., about 160 mg/day) administered in 2 daily doses (e.g., Q12H; e.g., 80 mg in the morning and 80 mg in the evening), wherein the subject prior to the administering the VB-201 has a PASI score that is from about 10 to about 20 (e.g., from about 14 to about 19, or from about 14.3 to about 18.5).

In other embodiments, the present disclosure provides a method of treating psoriasis, the method comprising administering to a subject in need thereof a therapeutically effective amount of VB-201, wherein the therapeutically effective amount is from about 80 mg/day to about 160 mg/day (e.g., about 160 mg/day) administered in 2 daily doses (e.g., Q12H; e.g., 80 mg in the morning and 80 mg in the evening), wherein the subject prior to the administering the VB-201 has psoriasis characterized by a body surface area (BSA) from about 10% to about 30% (e.g., from about 16% to about 24%).

In some examples according to any of the embodiments described herein, the VB-201 is administered to the subject for a treatment period of at least about 12 weeks, at least about 16 weeks, or at least about 24 weeks.

### BRIEF DESCRIPTION OF THE FIGURES

FIG. 1A is a bar graph illustrating that treatment of human CD 14⁺ monocytes with VB-201 (5 μg/ml) prior to chemotaxis assay decreases human monocyte migration towards various chemoattractants, such as MCP-1 (50 ng/ml), MIP-1a (50 ng/ml) or RANTES (100 ng/ml). FIG. 1B is a graph showing VB-201-mediated inhibition of LPS and Pam3CSK4 signaling in human monocytes.

FIG. 2 is a graph summarizing VB-201 clinical study design and dosing regimens.

FIG. 3 is a graph illustrating the distribution of patients at screening, randomization, participation, and follow-up of VB-201 clinical trial.

FIG. 4A is a bar graph illustrating a dose dependent change of mean of max target to background ratio (TBR) from baseline in most diseased segment (MDS) at 12 weeks upon VB-201 treatment. FIG. 4B is a bar graph comparing the changes of mean of max TBR from baseline in MDS at 12 weeks in the VB-201 treated group and the placebo treated group.

FIG. 5 is a bar graph illustrating the relationship between VB-201 trough levels and changes of mean of max target to background ratio (TBR) from baseline in most diseased segment (MDS) at 12 weeks.

FIG. 6 is a comparison of images illustrating reduced inflammation of the aorta and reduced inflammation surrounding the breast implants of a patient upon VB-201 treatment, using positron emission computed tomography (PET/CT) imaging quantifying 18-fluorodeoxyglucose (18-FDG) uptake as a TBR.

FIG. 7 is a bar graph illustrating the percentage of patients with psoriasis classified as “severe” (Physician Global Assessment (PGA) class 4) in placebo, 20 mg/day VB-201, and 80 mg/day VB-201 treated groups (see, Example 3) over a 12 week treatment period.

FIG. 8 is a bar graph showing the percentage of patients with psoriasis classified as “moderate/severe or worst (Patient Global Assessment (PtGA) class 3-5), in placebo, 20 mg/day VB-201, and 80 mg/day VB-201 treated groups (see, Example 3) over a 12 week treatment period.

FIG. 9 is a graph illustrating the percent change of PASI score compared to baseline PASI score for a subgroup of patients with a baseline PASI score from about 14.3 to about 18.5.

FIG. 10 is a bar graph showing the relationship between VB-201 trough levels and the percent changes of PASI score compared to baseline PASI score at 12 weeks.

FIG. 11 is a graph showing skin lesions in a patient who received VB-201 20 mg at baseline (A,B) and after 12 weeks of treatment (C,D).

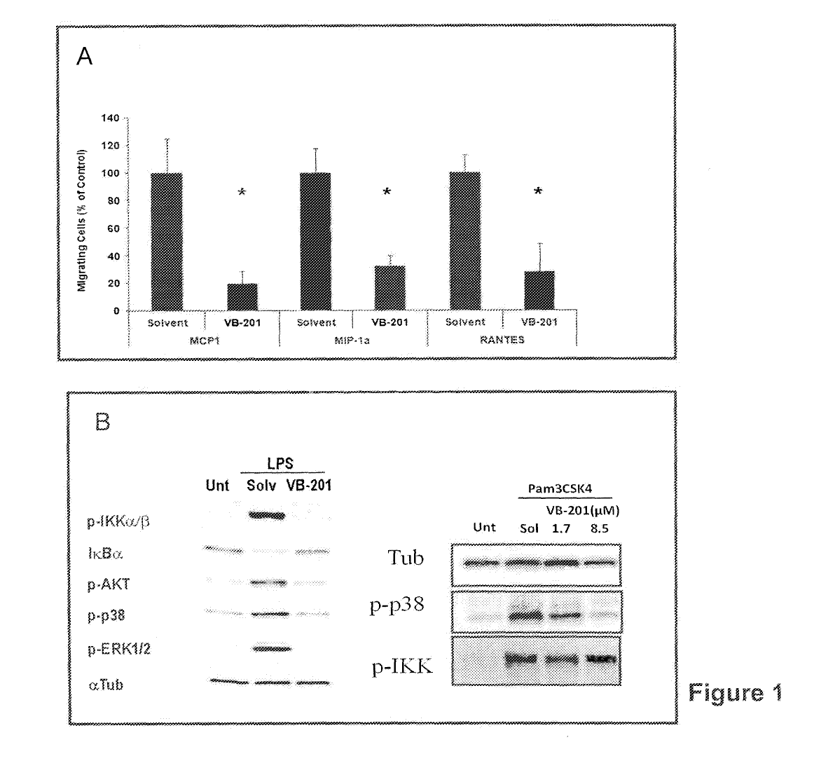

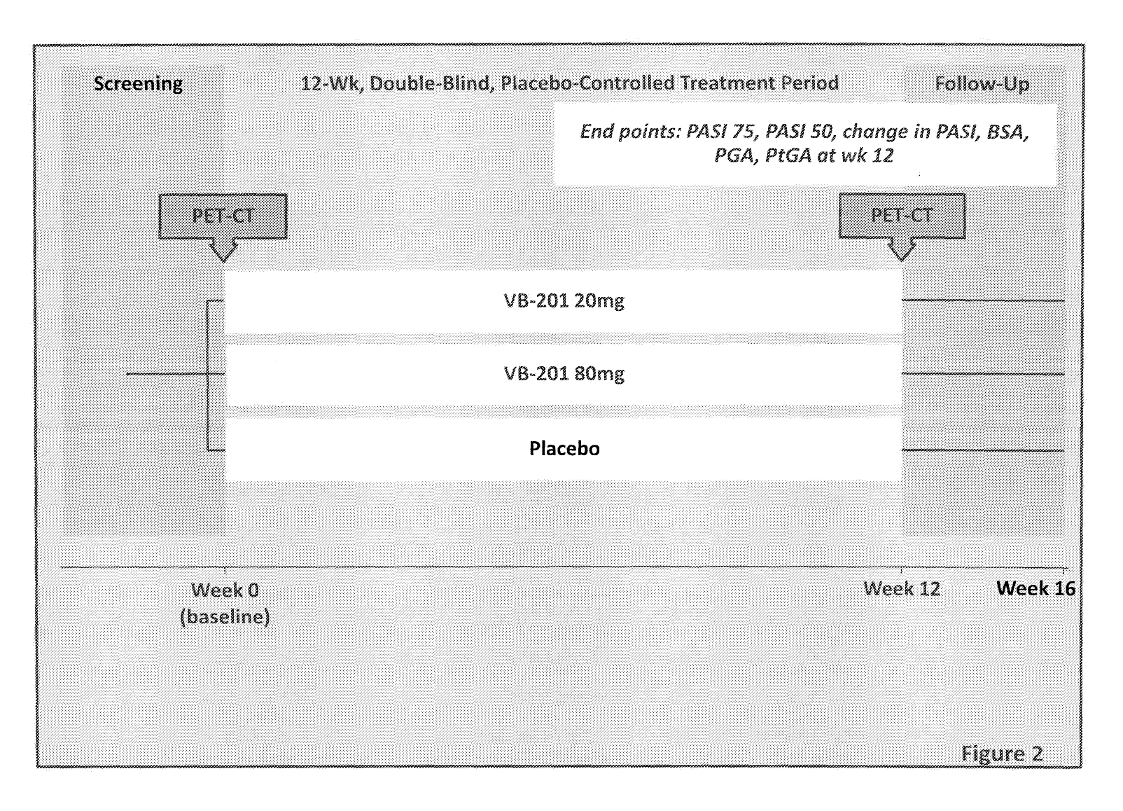

### DETAILED DESCRIPTION OF THE INVENTION

### Definitions

The term “psoriasis” means a disease or condition described by the medical name psoriasis vulgaris. There are different forms of psoriasis, with the most common form being plaque psoriasis. In some embodiments, the psoriasis is active plaque psoriasis. The severity of psoriasis can be categorized by the physician as clear, almost clear, mild, moderate, severe, and worse than severe. The severity of psoriasis described herein may be categorized according to the Physician's Global Assessment (PGA) score based on a 5-point scale with possible outcomes 0=clear, 1=almost clear, 2 mild, 3=moderate and 4=severe. The severity of psoriasis described herein may also be categorized according to the Patient's Psoriasis Global Assessment (PtGA) score based on a 6-point scale with possible outcomes 0=clear, 1=almost clear, 2=mild, 3=moderate, and 4=severe and 5=worst psoriasis has ever been. Unless specified, the terms “moderate” or “severe” psoriasis in any embodiments described herein may refer to either the PGA or the PtGA standard. In some embodiments, the psoriasis is moderate to severe, stable, active plaque psoriasis. In other examples, the psoriasis patient has a diagnosis of chronic plaque psoriasis for at least 6 months prior to administering the VB-201 to the subject. In some examples, the psoriasis (e.g., moderate to severe, stable, active plaque psoriasis) affects between about 10% to about 30% of the body surface area (BSA) of the subject prior to administering the VB-201. In other examples, the psoriasis (e.g., moderate to severe, stable, active plaque psoriasis) of the subject is characterized by a Psoriasis Area and Severity Index (PASI) score from about 10 to about 20 prior to administering the VB-201. In some examples, the active plaque psoriasis is characterized by a BSA of at least about 10% or at least about 20% but not more than about 30% prior to the administering the VB-201.

The term “PASI score” is used herein in accordance with its generally accepted meaning in the art (see, e.g., Feldman S R, Krueger G G. Psoriasis assessment tools in clinical trials. Ann. Rheum. Dis. 2005; 64 (Suppl II):ii65-ii68). An exemplary procedure to determine a “PASI score” includes the following steps: (a) divide the body into 4 areas (head, arms, trunk to groin, and legs to top of buttocks); (b) generate an average score for the erythema, thickness, and scale for each of the 4 areas (0=clear; 1=slight, 2=mild, 3=moderate, 4=severe); (c) sum scores of erythema, thickness and scale for each area; (d) generate a percentage for skin covered with psoriasis for each area and convert that to a 0-6 scale (0=0%; 1=<10%; 2=10-<30%; 3=30-<50%; 4=50-<70%; 5=70-<90%; 6=90-100%; (e) multiply score of item (c) with score of item (d) for each area and multiply the resulting number by 0.1 (for head), 0.2 (for arms), 0.3 (for trunk), and 0.4 (for legs), respectively, to obtain a score for each body area; (0 add the scores for all four body areas to get a PASI score in the range from 0 to 72.

The term “statin therapy” means administering a therapeutically effective amount of a statin according to regimens generally known to those of skill in the art (e.g., those indicated on packaging instructions for a respective statin drug product). For example the amount of a statin that would provide a therapeutic effect for which the statin is indicated, for example reducing a cholesterol level.

The term “statin” is used herein in accordance with its general meaning in the art. In one embodiment, the term “statin” in the context of this disclosure describes a class of drugs used to lower cholesterol levels by inhibiting the enzyme HMG-CoA reductase. This enzyme plays a central role in the production of cholesterol in the liver. Statins are widely used in the prevention and treatment of cardiovascular diseases (CVD). Exemplary statins include atorvastatin (Lipitor, Torvast), fluvastatin (Lescol), lovastatin (Mevacor, Altocor, Altoprev), pitavastatin (Livalo, Pitava), pravastatin (Pravachol, Selektine, Lipostat), rosuvastatin (Crestor) and simvastatin (Zocor, Lipex).

The term “therapeutically effective amount of VB-201” is the amount of VB-201 that is expected, e.g., based on a clinical study or practice by one of skill in the art (e.g., a physician) to provide, in at least a portion of the subjects receiving such a dose, a measurable therapeutic effect for which the VB-201 is indicated, for example reduced vascular inflammation, or improvement of psoriasis (e.g., PASI score). In any of the embodiments described herein, the therapeutically effective amount of VB-201 can be from about 1 mg/day to about 100 mg/day; about 101 mg/day to about 1 g/day; about 5 mg/day to about 240 mg/day; about 20 mg/day to about 240 mg/day; about 20 mg/day to about 160 mg/day; about 20 mg/day to about 80 mg/day; about 40 mg/day to about 240 mg/day; about 40 mg/day to about 160 mg/day; about 40 mg/day to about 80 mg/day; about 60 mg/day to about 240 mg/day; about 60 mg/day to about 160 mg/day; about 60 mg/day to about 80 mg/day; about 80 mg/day to about 240 mg/day; about 80 mg/day to about 160 mg/day; about 100 mg/day to about 240 mg/day; about 100 mg/day to about 160 mg/day; about 120 mg/day to about 240 mg/day; about 120 mg/day to about 160 mg/day; or about 160 mg/day to about 240 mg/day.

In any of the embodiments described herein, the therapeutically effective amount of VB-201 is about 20 mg/day; about 30 mg/day; about 40 mg/day; about 50 mg/day; about 60 mg/day; about 70 mg/day; about 80 mg/day; about 90 mg/day; about 100 mg/day; about 110 mg/day; about 120 mg/day; about 130 mg/day; about 140 mg/day; about 150 mg/day; about 160 mg/day; about 170 mg/day; about 180 mg/day; about 190 mg/day; about 200 mg/day; about 210 mg/day; about 220 mg/day; about 230 mg/day; or about 240 mg/day. In any of the embodiments described herein, the therapeutically effective amount of VB-201 may be about 80 mg/day; about 120 mg/day; or about 160 mg/day.

VB-201 is 1-hexadecyl-2-(4′-carboxy)butyl-sn-glycero-3-phosphocholine, also referred to as (R)-1-hexadecyl-2-(4′-carboxy)butyl-glycero-3-phosphocholine. The term VB-201 includes pharmaceutically acceptable salts or solvates (e.g., hydrates) thereof.

The phrase “pharmaceutically acceptable salt” refers to a charged species of the parent compound and its counter ion, which is typically used to modify the solubility characteristics of the parent compound and/or to reduce any significant irritation to an organism by the parent compound, while not abrogating the biological activity and properties of the administered compound. An example, without limitation, of a pharmaceutically acceptable salt would be a carboxylate anion and a cation such as, but not limited to, ammonium, sodium, potassium and the like.

The term “solvate” refers to a complex of variable stoichiometry (e.g., di-, tri-, tetra-, penta-, hexa-, and so on), which is formed by a solute (the compound of present embodiments) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

The term “treating” or “treatment” refers to administering a therapy in an amount, manner, and/or mode effective to prevent, or delay onset of, or amelioration of at least one symptom of a condition (e.g., vascular inflammation, inflammation associated with an implant or psoriasis). In any of the embodiments described herein, treating vascular inflammation includes reducing vascular inflammation. In any of the embodiments provided herein, treating inflammation associated with an implant includes reducing such inflammation.

### Treatment of Vascular Inflammation

The inventors have discovered in a phase 2 clinical trial (see Example 1) that VB-201 has a pronounced effect on vascular inflammation/atherosclerosis in human subjects, e.g., patients suffering from a chronic inflammatory or autoimmune disease, such as psoriasis. The human data is significant given the growing evidence linking increased cardiovascular events and elevated mortality in such patients. There is a widespread need for a targeted and safe oral medication for the long-term management of vascular inflammation in patients with autoimmune/inflammatory diseases, such as psoriasis.

### Method 1a

in various aspects, the present disclosure provides a method of treating (e.g., decreasing) vascular inflammation (e.g., vascular inflammation associated with atherosclerotic lesions) in a subject in need thereof (e.g., a human patient), the method comprising administering to the subject a therapeutically effective amount of VB-201 (e.g., from about 20 mg/day to about 160 mg/day). In some examples, the subject suffers from a chronic autoimmune/inflammatory disease (e.g., psoriasis). Examples of therapeutically effective amounts of VB-201 useful in Method 1a are described herein.

### Method 1b

Thus, in other aspects, the present disclosure provides a method of treating (e.g., decreasing) vascular inflammation (e.g., vascular inflammation associated with atherosclerotic lesions) in a subject in need thereof (e.g., a human patient), wherein the subject suffers from a chronic autoimmune or chronic inflammatory disease (e.g., psoriasis), the method comprising administering to the subject a therapeutically effective amount of VB-201 (e.g., from about 20 mg/day to about 160 mg/day). Examples of therapeutically effective amounts of VB-201 useful in Method 1b are described herein.

In some examples according to Methods 1a and 1b, the subject suffers from psoriasis. In other examples according to Methods 1a and 1b, the therapeutically effective amount of VB-201 (e.g., from about 20 mg/day to about 160 mg/day) is administered to the subject for at least about 8 weeks (e.g., at least about 10 weeks, or at least about 12 weeks).

### Method 2a

In other aspects, the present disclosure provides methods of treating (e.g., decreasing) vascular inflammation in a human subject suffering from a chronic autoimmune or chronic inflammatory disease (e.g., psoriasis), the method comprising administering to the subject a therapeutically effective amount of VB-201 (e.g., from about 20 mg/day to about 160 mg/day) for at least about 8 weeks (e.g., at least about 10 weeks, or at least about 12 weeks). In some examples, the vascular inflammation in the subject is reduced by, or at least by, about 4%, about 5%, about 6%, about 7%, about 8%, about 9%, about 10%, about 12%, about 15%, about 20%, or about 25% as compared to vascular inflammation in the subject prior to the administering the VB-201 to the subject (relative to baseline). Examples of therapeutically effective amounts of VB-201 useful in Method 2 are described herein. Alternate percentages of reduction of vascular inflammation in Method 2a are also described herein.

### Method 2b

In other aspects, the present disclosure provides methods of treating (e.g., decreasing) vascular inflammation in a human subject suffering from psoriasis, the method comprising administering to the subject a therapeutically effective amount of VB-201 (e.g., from about 20 mg/day to about 160 mg/day). In some examples, the subject is treated for at least about 8 weeks (e.g., at least about 10 weeks, or at least about 12 weeks or long term, e.g., at least 6 months, at least 1 year, at least 2 years, at least 5 years, or lifetime). In some examples, the subject is treated for about 8 weeks. In some examples, the subject is treated for less than 8 weeks (e.g., about 6 weeks, about 4 weeks, about 2 weeks, about 1 week, about 3 days). In some examples, the psoriasis is a mild, moderate, moderate to severe, severe, or worse than severe psoriasis. In some examples, the vascular inflammation in the subject is reduced by, or at least by, about 4%, about 5%, about 6%, about 7%, about 8%, about 9%, about 10%, about 12%, about 15%, about 20%, or about 25% as compared to vascular inflammation in the subject prior to the administering the VB-201 to the subject (relative to baseline). Examples of therapeutically effective amounts of VB-201 useful in Method 2b are described herein. Alternate percentages of reduction of vascular inflammation in Method 2b are also described herein.

According to the embodiments of Methods 1a, 1b, 2a, and 2b, in some embodiments, the vascular inflammation is associated with a cardiovascular disease, a peripheral vascular disease, a coronary artery disease, a cerebral vascular disease, a renal artery stenosis, an ischemic disease, or an aortic aneurism. In some embodiments, the vascular inflammation is associated with an ischemic heart disease, atherosclerosis, acute coronary syndrome, unstable angina, stable angina, or stroke. In preferred embodiments, the chronic autoimmune or chronic inflammatory disease is psoriasis.

According to the embodiments of Methods 1a, 1b, 2a, and 2b, in some embodiments, the vascular inflammation is associated with an inflammatory vascular disease. In some embodiments, the inflammatory vascular disease is Behcet's Disease, Buerger's Disease, Central Nervous System Vasculitis, Churg-Strauss Syndrome, Cryoglobulinemia, Giant Cell Arteritis, Henoch-Schonlein Purpura, Hypersensitivity Vasculitis, Kawasaki Disease, Microscopic Polyangiitis, Phlebitis, Polyarteritis Nodosa, Rheumatoid Vasculitis, Takayasu's Arteritis, or Wegener's Granulomatosis.

In any of the embodiments described herein, the vascular inflammation can be an inflammation of a carotid artery, an inflammation of aorta, or both.

According to the embodiments of Methods 1a, 1b, 2a, and 2b, in some embodiments, the subject is concurrently treated with an additional therapeutic agent. In some embodiments, the additional therapeutic agent is a steroid (e.g., a corticosteroid), a non-steroidal anti-inflammatory drug, an analgesic, an anti-atherosclerosis drug, an anti-proliferative agent, an immunosuppressant, a mucosal adjuvant, a growth factor, or a toxin. In some embodiments, the additional therapeutic agent is a HMGCoA reductase inhibitor (e.g., a statin), a cholesteryl ester transfer protein (CETP) inhibitor (e.g., avisimibe), a perixosome proliferative activated receptor (PPAR) agonist (e.g., fibrates), a cholesterol absorption inhibitor (e.g., ezetimibe), a squalene inhibitor, or any derivative and analog thereof. In some embodiments, the additional therapeutic agent is a Beta-2-glycoprotein I, ApoA-I Milano, nicotinic acid, a heat shock protein (HSP), or any derivative and analog thereof.

Non-limiting examples of steroids include, without limitation, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

Non-limiting examples of non-steroidal anti-inflammatory drugs include oxicams, such as piroxicam, isoxicam, tenoxicam, sudoxicam, and CP-14,304; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone.

Non-limiting examples of analgesics include aspirin and other salicylates (such as choline or magnesium salicylate), ibuprofen, ketoprofen, naproxen sodium, and acetaminophen.

Non-limiting examples of anti-proliferative agents include an alkylating agent such as a nitrogen mustard, an ethylenimine and a methylmelamine, an alkyl sulfonate, a nitrosourea, and a triazene; an antimetabolite such as a folic acid analog, a pyrimidine analog, and a purine analog; a natural product such as a vinca alkaloid, an epipodophyllotoxin, an antibiotic, an enzyme, a taxane, and a biological response modifier; miscellaneous agents such as a platinum coordination complex, an anthracenedione, an anthracycline, a substituted urea, a methyl hydrazine derivative, or an adrenocortical suppressant; or a hormone or an antagonist such as an adrenocorticosteroid, a progestin, an estrogen, an antiestrogen, an androgen, an antiandrogen, or a gonadotropin-releasing hormone analog. Specific examples of chemotherapeutic agents include, for example, a nitrogen mustard, an epipodophyllotoxin, an antibiotic, a platinum coordination complex, bleomycin, doxorubicin, paclitaxel, etoposide, 4-OH cyclophosphamide, and cisplatinum.

Immunosuppressant includes all drug molecules known to lessen the immune reaction in a subject (e.g.; a human subject). Immunosuppressant includes biologics, such as immunosuppressant antibodies, as well as non-biologic immunosuppressant. Non-limiting examples of non-biologic immunosuppressant include antimetabolites, such as folic acid analogues (e.g., methotrexate); purine analogues (e.g., azathioprine and mercaptopurine); pyrimidine analogues, protein synthesis inhibitors, cytotoxic antibiotics (e.g., dactinomycin, anthra, cyclines, mitomycin C, bleomycin, and mithramycin); calcineurin inhibitors (CNI) (e.g., cyclosporin, myriocin, tacrolimus, sirolimus), mycophenolate, and fingolimod.

Growth factors are hormones, which have numerous functions. Non-limiting examples of growth factors include insulin-like growth factor-1 (IGF-1), transforming growth factor-11 (TGF-β) a bone morphogenic protein (BMP) and the like.

Non-limiting examples of toxins include the cholera toxin, which also can serve as an adjuvant.

HMGCoA reductase inhibitors (e.g., statins) are well known drugs that effectively reduce LDL-cholesterol levels by inhibiting the enzyme that regulates the rate of cholesterol production and increasing the clearance of LDL-cholesterol present in the blood by the liver. Non-limiting examples of commonly prescribed statins include atorvastatin, fluvastatin, lovastatin, pravastatin and simvastatin.

Non-limiting examples of Proliferative Activated Receptor (PPAR) agonists, include fibrates, such as bezafibrate, gemfibrozil and fenofibrate.

Effect of VB-201 on Vascular Inflammation in Patients Treated with Statins

The inventors have discovered in a phase 2 clinical trial (see Example 1) that VB-201 has a pronounced effect on vascular inflammation in subjects (e.g., human patients) who were treated with a statin prior to receiving VB-201 treatment and/or continued statin therapy after the onset of VB-201 treatment (i.e., were on statin therapy at the beginning of the trial and continued treatment throughout the trial). Statins themselves can reduce vascular inflammation. Therefore, it was expected that patients undergoing statin therapy may not experience the same improvement of vascular inflammation due to VB-201 treatment as patients who did not undergo statin therapy and may experience a reduced benefit from VB-210. Unexpectedly, this was not the case. Patients undergoing statin therapy prior to the onset of VB-201 treatment experienced a similar benefit from VB-201 with respect to vascular inflammation (e.g., improvement over baseline during a 12-week treatment period) than patients not undergoing statin therapy. Thus, VB-201 can further improve vascular inflammation in patients undergoing statin therapy prior to the onset of treatment with VB-201.

In some instances, the effect seen for VB-201 in patients undergoing statin therapy was even slightly better than in patients not being treated with statins indicating a possible synergy between statins and VB-201 with respect to decreasing vascular inflammation.

Thus, in some examples according to any one of the embodiments of Methods 1a, 1b, 2a, and 2b described herein, the subject underwent statin therapy prior to administering the VB-201 (e.g., wherein a therapeutically effective amount of a statin is administered to the subject during a time period immediately prior to first administering the VB-201). In other examples according to any one of the embodiments of Methods 1a, 1b, 2a, and 2b, the subject is concomitantly treated with a statin.

### Method 3

In various aspects, the current disclosure further provides a method of treating (e.g., decreasing) vascular inflammation, the method comprising administering to a subject in need thereof a therapeutically effective amount of VB-201 (e.g., from about 20 mg/day to about 160 mg/day), wherein the subject underwent statin therapy prior to administering the VB-201 (e.g., wherein a therapeutically effective amount of a statin is administered to the subject during a time period immediately prior to first administering the VB-201).

In one example according to any one of the embodiments of Methods 1a, 1b, 2a, 2b, and 3 described herein, the subject underwent statin therapy (i.e., administration of a therapeutically effective amount of a statin) for at least about 2 weeks prior to first administering the VB-201. In other examples according to any one of the embodiments described herein, the subject underwent statin therapy for at least about 1 month prior to the administering of the VB-201. In other examples according to any one of the embodiments described herein, the subject underwent statin therapy for at least about 2 months prior to the administering of the VB-201. In other examples according to any one of the embodiments described herein, the subject underwent statin therapy for at least about 3 months prior to the administering of the VB-201. In other examples according to any one of the embodiments described herein, the subject underwent statin therapy for at least about 4 months prior to first administering the VB-201. In other example according to any one of the embodiments described herein, the subject underwent statin therapy for at least about 5 months prior to the administering the VB-201. In other examples according to any one of the embodiments described herein, the subject underwent statin therapy for at least about 6 months prior to the administering the VB-201. In yet other examples according to any one of the embodiments described herein, the subject underwent statin therapy from about 1 week to about 2 months prior to first administering the VB-201.

In some examples according to any one of the embodiments of Methods 1a, 1b, 2a, 2b, and 3, the subject continues undergoing statin therapy after the onset of treating the subject with the VB-201 (i.e., after first administering the VB-201 to the subject).

In some examples according to any one of the embodiments of Methods 1a, 1b, 2a, 2b, and 3, the statin therapy comprises treating the subject with a statin described herein (e.g., atorvastatin, fluvastatin, lovastatin, or simvastatin) below a maximum acceptable dosage. In some examples, the maximum acceptable dosage is a maximum recommended dosage a recognized organization or agency (e.g., the U.S. Food and Drug Administration (FDA) and/or the European Medicines Agency). In some examples, the statin therapy comprises treating the subject with a statin described herein (e.g., atorvastatin, fluvastatin, lovastatin, or simvastatin) in about 80 mg/day, about 75 mg/day, about 70 mg/day, about 65 mg/day, about 60 mg/day, about 55 mg/day, about 50 mg/day, about 45 mg/day, about 40 mg/day, about 35 mg/day, about 30 mg/day, about 25 mg/day, about 20 mg/day, about 15 mg/day, about 10 mg/day, or about 5 mg/day.

In some examples according to any one of the embodiments of Methods 1a, 1b, 2a, 2b, and 3 described herein, the vascular inflammation is measured using positron emission computed tomography (PET/CT) imaging quantifying 18-fluorodeoxyglucose (18-FDG) uptake as a target to background ratio (TBR), e.g., as described herein, or in Example 8 of WO2011/083465.

### Method 4

Thus, the present invention provides a method of decreasing vascular inflammation in a subject, the method comprising administering to a subject a therapeutically effective amount of VB-201, wherein the therapeutically effective amount is about 20 mg/day to about 160 mg/day (e.g., 20 mg/day to about 80 mg/day, or about 80 mg/day to about 160 mg/day). In some examples the vascular inflammation after administering the therapeutically effective amount to the subject (e.g., for at least about 12 weeks) is reduced by, or at least by, about 4%, about 5%, about 6%, about 7%, about 8%, about 9%, about 10%, about 12%, about 15%, about 20%, or about 25% as compared to vascular inflammation in the subject prior to the administering the VB-201 to the subject (relative to baseline). In some examples, the vascular inflammation is measured using positron emission computed tomography (PET/CT) imaging quantifying 18-fluorodeoxyglucose (18-FDG) uptake as a target to background ratio (TBR) (e.g., as described herein, or in Example 8 of WO2011/083465).

In some examples according to any one of the embodiments of Methods 1a, 1b, 2a, 2b, 3, and 4 described herein, vascular inflammation in the subject is decreased within a relatively short treatment period (e.g., not more than about 12 weeks) during which vascular inflammation is reduced by a certain percentage (e.g., at least about 10% compared to baseline). In some examples according to any of the embodiments described herein, the vascular inflammation (e.g., after administering the therapeutically effective amount to the subject for about 12 weeks or about 24 weeks) is reduced by at least about 5% when compared to the vascular inflammation prior to the administering (base line). In other examples according to any of the embodiments described herein, the vascular inflammation (e.g., after administering the therapeutically effective amount to the subject for about 12 weeks or about 24 weeks) is reduced by at least about 6% when compared to the vascular inflammation prior to the administering. In other examples according to any of the embodiments described herein, the vascular inflammation (e.g., after administering the therapeutically effective amount to the subject for about 12 weeks or about 24 weeks) is reduced by at least about 8% when compared to the vascular inflammation prior to the administering. In other examples according to any of the embodiments described herein, the vascular inflammation (e.g., after administering the therapeutically effective amount to the subject for about 12 weeks or 24 weeks) is reduced by, or at least by, about 4%, about 5%, about 6%, about 7%, about 8%, about 9%, about 10%, about 12%, about 15%, about 20%, or about 25% when compared to the vascular inflammation prior to the administering. In other examples according to any of the embodiments described herein, the vascular inflammation (e.g., after administering the therapeutically effective amount to the subject for about 12 weeks or 24 weeks) is reduced by at least about 12% when compared to the vascular inflammation prior to the administering. In other examples according to any of the embodiments described herein, the vascular inflammation (e.g., after administering the therapeutically effective amount to the subject for about 12 weeks or 24 weeks) is reduced by at least about 14% when compared to the vascular inflammation prior to the administering. In other examples according to any of the embodiments described herein, the vascular inflammation (e.g., after administering the therapeutically effective amount to the subject for about 12 weeks or 24 weeks) is reduced by at least about 16% when compared to the vascular inflammation prior to the administering. In other examples according to any of the embodiments described herein, the vascular inflammation (e.g., after administering the therapeutically effective amount to the subject for about 12 weeks or 24 weeks) is reduced by at least about 18% when compared to the vascular inflammation prior to the administering. In other examples according to any of the embodiments described herein, the vascular inflammation (e.g., after administering the therapeutically effective amount to the subject for about 12 weeks or 24 weeks) is reduced by at least about 20% when compared to the vascular inflammation prior to the administering.

In some examples according to any one of the embodiments of Methods 1a, 1b, 2a, 2b, 3, and 4 described herein, the therapeutically effective amount is administered to the subject for at least about 8 weeks. In other examples according to any one of the embodiments described herein, the therapeutically effective amount is administered to the subject for about 8 weeks or not more than about 8 weeks. In other examples according to any one of the embodiments described herein, the therapeutically effective amount is administered to the subject for at least about 12 weeks. In other examples according to any one of the embodiments described herein, the therapeutically effective amount is administered to the subject for about 12 weeks or not more than about 12 weeks. In other examples according to any one of the embodiments described herein, the therapeutically effective amount is administered to the subject for at least about 14 weeks. In other examples according to any one of the embodiments described herein, the therapeutically effective amount is administered to the subject for about 14 weeks or not more than about 14 weeks. In other examples according to any one of the embodiments described herein, the therapeutically effective amount is administered to the subject for at least about 16 weeks. In other examples according to any one of the embodiments described herein, the therapeutically effective amount is administered to the subject for about 16 weeks or not more than about 16 weeks. In other examples, the therapeutically effective amount is administered to the subject for at least about 18 weeks. In other examples, the therapeutically effective amount is administered to the subject for about 18 weeks or not more than about 18 weeks. In other examples, the therapeutically effective amount is administered to the subject for at least about 20 weeks. In other examples, the therapeutically effective amount is administered to the subject for about 20 weeks or not more than about 20 weeks. In other examples, the therapeutically effective amount is administered to the subject for at least about 24 weeks. In other examples, the therapeutically effective amount is administered to the subject for about 24 weeks or not more than about 24 weeks.

In other examples according to any one of the embodiments of Methods 1a, 1b, 2a, 2b, 3, and 4 described herein, the subject has an elevated high sensitivity C-reactive protein (hs-CRP) level prior to first administering the VB-201.

In other examples according to any one of the embodiments of Methods 1a, 1b, 2a, 2b, 3, and 4 described herein, the subject does not have an elevated high sensitivity C-reactive protein (hs-CRP) level prior to first administering the VB-201.

In other examples according to any one of the embodiments of Methods 1a, 1b, 2a, 2b, 3, and 4 described herein, the subject has not been on a stable high dose of statin (e.g., ≧20 mg/day atorvastatin; or ≧10 mg/day rosuvastatin; or ≧40 mg/day simvastatin). In another example according to any one of the described embodiments, the subject underwent statin therapy with less than a high dose of statin (e.g., less than 20 mg/day atorvastatin; or less than 10 mg/day rosuvastatin; or less than 40 mg/day simvastatin). In another example according to any one of the described embodiments, the subject underwent statin therapy for less than 3 months prior to first administering the VB-201.

In some examples according to any one of the embodiments of Methods 1a, 1b, 2a, 2b, 3, and 4 described herein, the vascular inflammation is inflammation of a carotid artery. In another embodiment, the vascular inflammation is inflammation of an aorta.

In some examples according to any one of the embodiments of Methods 1a, 1b, 2a, 2b, 3, and 4 described herein, the vascular inflammation is associated with atherosclerosis (i.e., the subject suffers from atherosclerosis). In another embodiment according to any of the embodiments described herein, the vascular inflammation is associated with cardiovascular disease (i.e., the subject suffers from a cardiovascular disease). In yet other examples according to any one of the embodiments described herein, the vascular inflammation is associated with psoriasis (i.e., the subject suffers from psoriasis).

In some examples according to any one of the embodiments of Methods 1a, 1b, 2a, 2b, 3, and 4 described herein, the therapeutically effective amount is from about 5 mg/day to about 240 mg/day, or from about 10 mg/day to about 240 mg/day. In other examples according to any one of the described embodiments, the therapeutically effective amount is from about 20 mg/day to about 240 mg/day, or from about 40 mg/day to about 240 mg/day. In other examples according to any one of the described embodiments, the therapeutically effective amount is from about 20 mg/day to about 200 mg/day, or from about 20 mg/day to about 180 mg/day. In other examples according to any one of the described embodiments, the therapeutically effective amount is from about 10 mg/day to about 160 mg/day, or from about 20 mg/day to about 160 mg/day. In other examples according to any one of the embodiments described herein, the therapeutically effective amount is from about 40 mg/day to about 160 mg/day.

In other examples according to any one of the embodiments described herein, the therapeutically effective amount is from about 40 mg/day to about 160 mg/day, or from about 50 mg/day to about 160 mg/day. In other examples according to any one of the embodiments described herein, the therapeutically effective amount is from about 60 mg/day to about 160 mg/day. In other examples according to any one of the described embodiments, the therapeutically effective amount is from about 80 mg/day to about 160 mg/day. In other examples according to any one of the embodiments described herein, the therapeutically effective amount is about 100 mg/day to about 160 mg/day, or from about 120 mg/day to about 160 mg/day. In other examples according to any one of the embodiments described herein, the therapeutically effective amount is about 80 mg/day. In other examples according to any one of the embodiments described herein, the therapeutically effective amount is about 120 mg/day. In other examples according, to any one of the embodiments described herein, the therapeutically effective amount is about 160 mg/day.

In other examples according to any one of the embodiments described herein, the therapeutically effective amount is from about 20 mg/day to about 120 mg/day. In other examples according to any one of the embodiments described herein, the therapeutically effective amount is from about 20 mg/day to about 100 mg/day, in other examples according to any one of the embodiments described herein, the therapeutically effective amount is from about 20 mg/day to about 120 mg/day. In some examples according to any one of the embodiments described herein, the therapeutically effective amount is from about 20 mg/day to about 80 mg/day. In some examples according to any one of the embodiments described herein, the therapeutically effective amount is from about 40 mg/day to about 80 mg/day.

The inventors have further discovered in human clinical studies that, in humans, VB-201 is well tolerated up to a dose of about 80 mg/day, but larger doses (e.g., 160 mg/day) are associated with certain gastrointestinal events (G-I intolerance, mainly nausea and vomiting, but no laboratory abnormalities or serious G-I adverse events) when administered a single daily dose. Surprisingly, such gastrointestinal events were largely avoided when the higher dose (e.g., 160 mg/day) was administered in multiple sub-doses (e.g., two sub-doses) several hours (e.g., 12 hours) apart from each other, e.g., when each sub-dose was about 80 mg or less.

Thus, in some examples according to any one of the embodiments described herein, wherein the VB-201 is administered at a daily dose of more than about 80 mg/day (e.g., 120 mg/day or 160 mg/day) then the total VB-201 dose is administered in at least two daily sub-doses, e.g., 2, 3, 4, 5, or 6 daily sub-doses, e.g., one in the morning and one in the evening with about 12 hours between sub-doses, e.g., every 12 hours (Q12H) or every 12±2 hours (i.e., about 10 hours to about 14 hours). In some examples according to any of the embodiments described herein, when the VB-201 is administered at a daily dose of about 120 mg/day, the VB-201 is administered in two sub-doses of 40 mg and 80 mg (e.g., 40 mg in the morning and 80 mg in the evening, or 80 mg in the morning and 40 mg in the evening) or in two sub-doses of 60 mg each. In other examples according to any of the embodiments described herein, when the VB-201 is administered at a daily dose of about 160 mg/day, the VB-201 is administered in two equal sub-doses of about 80 mg each (e.g., Q12H or every 12±2 hours).

In one embodiment according to any of the embodiments described herein, the therapeutically effective amount is about 20 mg/day administered to the subject in 1 or 2 daily doses. In other examples according to any of the embodiments described herein, the therapeutically effective amount is about 20 mg/day administered to the subject in a single daily dose. In other examples according to any of the embodiments described herein, the therapeutically effective amount is about 40, 60, or 80 mg/day administered to the subject in 1 or 2 daily doses. In other examples according to any of the embodiments described herein, the therapeutically effective amount is about 80 mg/day administered to the subject in a single daily dose.

### Method 5a

In some embodiments, the present disclosure provides a method of treating (e.g., decreasing) vascular inflammation (e.g., vascular inflammation associated with atherosclerotic lesions) in a subject in need thereof (e.g., a human patient), the method comprising administering to the subject a therapeutically effective amount of VB-201, wherein the therapeutically effective amount of VB-201 is from about 120 mg/day to about 160 mg/day (e.g., 160 mg/day), and wherein the therapeutically effective amount is administered to the subject in at least two daily sub-doses, wherein each sub-dose is 80 mg or less.

In some examples of Method 5a, the total VB-201 dose is administered in two sub-doses, e.g., one in the morning and one in the evening with about 12 hours between sub-doses, e.g., every 12 hours (Q12H). In some examples according to any of the embodiments of Method 5a described herein, when the VB-201 is administered at a daily dose of about 120 mg/day, the VB-201 is administered in two sub-doses of 40 mg and 80 mg (e.g., 40 mg in the morning and 80 mg in the evening, or 80 mg in the morning and 40 mg in the evening), or is administered in two equal sub-doses of 60 mg each. In other examples according to any of the embodiments of Method 5a described herein, when the VB-201 is administered at a daily dose of about 160 mg/day, the VB-201 is administered in two equal sub-doses of about 80 mg each (e.g., Q12H).

Representative chronic autoimmune/inflammatory diseases include, for example, idiopathic inflammatory diseases or disorders, chronic inflammatory diseases or disorders, acute inflammatory diseases or disorders, autoimmune diseases or disorders, infectious diseases or disorders, inflammatory malignant diseases or disorders, inflammatory transplantation-related diseases or disorders, inflammatory degenerative diseases or disorders, diseases or disorders associated with a hypersensitivity, inflammatory cardiovascular diseases or disorders (e.g., as described herein), inflammatory cerebrovascular diseases or disorders, peripheral vascular diseases or disorders, inflammatory glandular diseases or disorders, inflammatory gastrointestinal diseases or disorders, inflammatory cutaneous diseases or disorders, inflammatory hepatic diseases or disorders, inflammatory neurological diseases or disorders, inflammatory musculo-skeletal diseases or disorders, inflammatory renal diseases or disorders, inflammatory reproductive diseases or disorders, inflammatory systemic diseases or disorders, inflammatory connective tissue diseases or disorders, inflammatory tumors, necrosis, inflammatory implant-related diseases or disorders, inflammatory aging processes, immunodeficiency diseases or disorders, proliferative diseases and disorders, and inflammatory pulmonary diseases or disorders.

Non-limiting examples of hypersensitivities include Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity, delayed type hypersensitivity, helper T lymphocyte mediated hypersensitivity, cytotoxic T lymphocyte mediated hypersensitivity, TH1 lymphocyte mediated hypersensitivity, and TH2 lymphocyte mediated hypersensitivity.

Non-limiting examples of cerebrovascular diseases or disorders include stroke, cerebrovascular inflammation, cerebral hemorrhage and vertebral arterial insufficiency.

Non-limiting examples of peripheral vascular diseases or disorders include gangrene, diabetic vasculopathy, ischemic bowel disease, thrombosis, diabetic retinopathy and diabetic nephropathy.

Non-limiting examples of autoimmune diseases or disorders include all of the diseases caused by an immune response such as an autoantibody or cell-mediated immunity to an autoantigen and the like. Representative examples are chronic rheumatoid arthritis, juvenile rheumatoid arthritis, systemic lupus erythematosus, scleroderma, mixed connective tissue disease, polyarteritis nodosa, polymyositis/dermatomyositis, Sjogren's syndrome, Bechet's disease, multiple sclerosis, autoimmune diabetes, Hashimoto's disease, psoriasis, primary myxedema, pernicious anemia, myasthenia gravis, chronic active hepatitis, autoimmune hemolytic anemia, idiopathic thrombocytopenic purpura, uveitis, vasculitides and heparin induced thrombocytopenia.

Non-limiting examples of inflammatory glandular diseases or disorders include pancreatic diseases or disorders, Type I diabetes, thyroid diseases or disorders, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome.

Non-limiting examples of inflammatory gastrointestinal diseases or disorders include colitis, ileitis, Crohn's disease, chronic inflammatory intestinal disease, inflammatory bowel syndrome, inflammatory bowel disease, celiac disease, ulcerative colitis, an ulcer, a skin ulcer, a bed sore, a gastric ulcer, a peptic ulcer, a buccal ulcer, a nasopharyngeal ulcer, an esophageal ulcer, a duodenal ulcer and a gastrointestinal ulcer.

Non-limiting examples of inflammatory cutaneous diseases or disorders include acne, an autoimmune bullous skin disease, pemphigus vulgaris, bullous pemphigoid, pemphigus foliaceus, contact dermatitis and drug eruption.

Non-limiting examples of inflammatory hepatic diseases or disorders include autoimmune hepatitis, hepatic cirrhosis, non-alcoholic steatohepatitis (NASH), and biliary cirrhosis.

Non-limiting examples of inflammatory neurological diseases or disorders include multiple sclerosis, Alzheimer's disease, ‘Parkinson's disease, myasthenia gravis, motor neuropathy, Guillain-Barre syndrome, autoimmune neuropathy, Lambert-Eaton myasthenic syndrome, paraneoplastic neurological disease or disorder, paraneoplastic cerebellar atrophy, non-paraneoplastic stiff man syndrome, progressive cerebellar atrophy, Rasmussen's encephalitis, amyotrophic lateral sclerosis, Sydeham chorea, Gilles de la Tourette syndrome, autoimmune polyendocrinopathy, dysimmune neuropathy, acquired neuromyotonia, arthrogryposis multiplex, Huntington's disease, AIDS associated dementia, amyotrophic lateral sclerosis (ALS), multiple sclerosis, stroke, an inflammatory retinal disease or disorder, an inflammatory ocular disease or disorder, optic neuritis, spongiform encephalopathy, migraine, headache, cluster headache, and stiff-man syndrome.

Non-limiting examples of inflammatory connective tissue diseases or disorders include autoimmune myositis, primary Sjogren's syndrome, smooth muscle autoimmune disease or disorder, myositis, tendinitis, a ligament inflammation, chondritis, a joint inflammation, a synovial inflammation, carpal tunnel syndrome, arthritis, rheumatoid arthritis, osteoarthritis, ankylosing spondylitis, a skeletal inflammation, an autoimmune ear disease or disorder, and an autoimmune disease or disorder of the inner ear.

Non-limiting examples of inflammatory renal diseases or disorders include autoimmune interstitial nephritis and/or renal cancer.

Non-limiting examples of inflammatory reproductive diseases or disorders include repeated fetal loss, ovarian cyst, or a menstruation associated disease or disorder.

Non-limiting examples of inflammatory systemic diseases or disorders include systemic lupus erythematosus, systemic sclerosis, septic shock, toxic shock syndrome, and cachexia.

Non-limiting examples of infectious disease or disorder include chronic infectious diseases or disorders, a subacute infectious disease or disorder, an acute infectious disease or disorder, a viral disease or disorder, a bacterial disease or disorder, a protozoan disease or disorder, a parasitic disease or disorder, a fungal disease or disorder, a mycoplasma disease or disorder, gangrene, sepsis, a prion disease or disorder, influenza, tuberculosis, malaria, acquired immunodeficiency syndrome, and severe acute respiratory syndrome.

Non-limiting examples of inflammatory transplantation-related diseases or disorders include graft rejection, chronic graft rejection, subacute graft rejection, acute graft rejection hyperacute graft rejection, and graft versus host disease or disorder. Exemplary implants include a prosthetic implant, a breast implant, a silicone implant, a dental implant, a penile implant, a cardiac implant, an artificial joint, a bone fracture repair device, a bone replacement implant, a drug delivery implant, a catheter, a pacemaker, an artificial heart, an artificial heart valve, a drug release implant, an electrode, and a respirator tube.

Non-limiting examples of inflammatory tumors include a malignant tumor, a benign tumor, a solid tumor, a metastatic tumor and a non-solid tumor.

Non-limiting examples of inflammatory pulmonary diseases or disorders include asthma, allergic asthma, emphysema, chronic obstructive pulmonary disease or disorder, sarcoidosis, bronchitis, and pulmonary fibrosis (e.g. idiopathic pulmonary fibrosis [IPF]).

An example of a proliferative disease or disorder is cancer.

Treatment of Inflammation Associated with an Implant

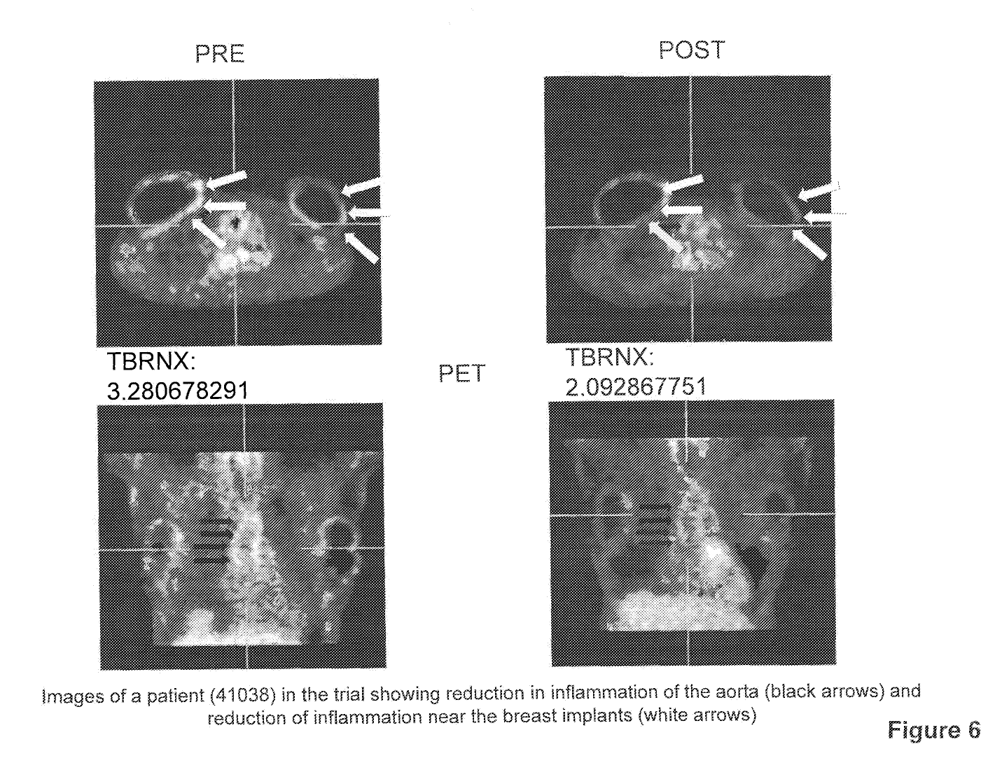

The inventors have also unexpectedly discovered in a phase 2 clinical trial that VB-201 has a pronounced effect on inflammation associated with implants (e.g., breast implants, See FIG. 6).

### Method 5b

Thus, in various embodiments, the present disclosure also provides methods of treating inflammation associated with an implant in a subject in need thereof (e.g., a human patient), The methods comprising administering to the subject a therapeutically effective amount of VB-201 (e.g., about 20 mg/day to about 160 mg/day). In some embodiments, the inflammation associated with an implant is a reactive inflammation, for example, a soft tissue inflammation. In some embodiments, the inflammation associated with an implant is a local inflammation (i.e., inflammation near an implant) or systemic inflammatory reaction. In some embodiments, the implant is a silicone, a saline, a metal, a plastic, or a polymeric implant. In some embodiments, the implant is a cosmetic implant, a prosthetic implant, a subdermal implant, a transdermal implant, a bone replacement implant, or a bone fracture repair device. In some embodiments, the implant is a drug delivery implant or a drug release implant. In some embodiments, the implant is an artificial joint, an artificial heart, an artificial heart valve, a testicular prosthesis, a breast implant, a dental implant, an ocular implant (e.g., artificial lens), a cochlear implant, a penile implant, a cardiac implant, a catheter, an implantable urinary continence device, a pacemaker, an electrode, a Hernia support devices (e.g., nets), or a respirator tube. In some embodiments, the implant is a breast implant.

In some examples according to any of the embodiments of Method 5b, the methods comprise treating or reducing inflammation associated with the implant. In some examples, the methods reduce inflammation associated with the implant in the subject after administering the therapeutically effective amount to the subject (e.g., for at least about 12 weeks). In some examples, the inflammation is reduced by, or at least by, about 4%, about 5%, about 6%, about 7%, about 8%, about 9%, about 10%, about 12%, about 15%, about 20%, or about 25% as compared to inflammation in the subject prior to the administering the VB-201 to the subject (relative to baseline). In some examples, the inflammation is measured using positron emission computed tomography (PET/CT) imaging quantifying 18-fluorodeoxyglucose (18-FDG) uptake as a target to background ratio (TBR) (e.g., as described herein, or in Example 8 of WO2011/083465).

In any of the embodiments according to Method 5b, the therapeutically effective amount of VB-201 may be about 80 mg/day, about 120 mg/day, or about 160 mg/day. Other suitable daily dosages of VB-201 are described herein. In some examples, the therapeutically effective amount of VB-201 is administered to the subject in 1 or 2 daily sub-doses. In some examples, the 2 daily sub-doses are administered at different times of a day, for example, about 10 hours apart, or about 12 hours apart, or one in the morning and one in the evening. In some examples, the 2 daily dosages are about equal in amounts (e.g., a 160 mg/day may be administered by two sub-doses of about 80 mg each), or different in amounts (e.g., a 120 mg/day may be administered by one sub-dose of about 80 mg and one sub-dose of 40 mg). In some examples of Method 5b, the therapeutically effective amount of VB-201 is administered to the subject for a relatively short treatment period of less than about 12 weeks, less than about 8 weeks, or less than about 4 weeks. In other examples, the therapeutically effective amount of VB-201 is administered to the subject for a treatment period of at least 4 weeks, for example, about 4 weeks, about 8 weeks, about 12 weeks, about 16 weeks, about 24 weeks, or more than about 24 weeks.

In some embodiments, the invention provides methods of treating a subject having inflammation associated with an implant, wherein the subject also is in need of treatment for vascular inflammation by administering VB-201 to the subject. In some embodiments, the subject also is in need of treatment of an autoimmune disorder, such as psoriasis. In some embodiments, the implant is a breast implant. In some embodiments, the inflammation associated with an implant is a local inflammation or a systemic inflammatory reaction.

Treatment of Psoriasis

In various aspects, the present disclosure provides methods of treating psoriasis. Unexpectedly, the inventors have discovered that patients with a particular severity of the disease at baseline, respond particularly well to treatment with VB-201. For example, patients with a baseline Psoriasis Area and Severity Index (PASI) score from about 10 to about 20 (e.g., from about 14 to about 19, or from about 14.3 to about 18.5), or psoriasis characterized by a body surface area (BSA) from about 10% to about 30% (e.g., from about 16% to about 24%), responded particularly well to VB-201 treatment. Furthermore, patients not previously treated with immune-suppressants or biologic psoriasis medications, as well as patients with an elevated baseline high sensitivity C-reactive protein (CRP) responded particularly well to VB-201 treatment. Moreover, patients with a particular duration of psoriasis (e.g., at least 6 months) responded particularly well to VB-201 treatment.

Thus, in various embodiments, the present disclosure provides a method of treating moderate to severe psoriasis in a subject in need thereof, the method comprising administering to the subject a therapeutically effective amount of VB-201 for a treatment period, wherein: a) the subject has a PASI score of about 10 to about 20 prior to the treatment period; b) the subject has a BSA of 10% to 30% prior to the treatment period; c) the subject was not treated with an anti-psoriatic biologic or an immunosuppressant drug prior to the treatment period; or any combinations thereof.

### Method 6

In some embodiments, the present disclosure provides methods of treating severe psoriasis, wherein severe psoriasis is psoriasis of category 4 according to the Physician Global Assessment (PGA) scale. In some embodiments, the method comprises administering to a subject in need thereof a therapeutically effective amount of VB-201 as defined herein (e.g., the therapeutically effective amount is from about 80 mg/day to about 160 mg/day) for a treatment period. In some examples, the severe psoriasis improves to moderate, mild, almost clear, or no psoriasis (psoriasis of categories 0-3 according to PGA scale) during the treatment period. In some examples according to Method 6, the therapeutically effective amount is administered to the subject for a treatment period of at least about 8 weeks (e.g., 8 weeks, 12 weeks, 16 weeks, 20 weeks, or 24 weeks).

### Method 7

In other embodiments, the present disclosure provides methods of treating moderate to severe psoriasis, wherein moderate to severe psoriasis is psoriasis of categories 3 and 4 according to the Physician Global Assessment (PGA) scale. The method comprises administering to a subject in need thereof a therapeutically effective amount of VB-201, wherein the therapeutically effective amount is defined herein (e.g., the therapeutically effective amount is from about 80 mg/day to about 160 mg/day). The therapeutically effective amount is administered to the subject for a treatment period of at least about 8 weeks (e.g., 8 weeks, 12 weeks, 16 weeks, 20 weeks, or 24 weeks). In some examples, the psoriasis improves to mild, almost clear or no psoriasis (psoriasis of categories 0-2 according to PGA scale) during the treatment period.

### Method 8a

In other embodiments, the present disclosure provides a method of treating moderate, severe, or worse than severe psoriasis, which is psoriasis of categories 3 to 5 according to the Patient Global Assessment (PtGA) scale. The method comprises administering to a subject in need thereof a therapeutically effective amount of VB-201, wherein the therapeutically effective amount is defined herein (e.g., the therapeutically effective amount is from about 80 mg/day to about 160 mg/day) for a treatment period of at least about 8 weeks (e.g., 8 weeks, 12 weeks, 16 weeks, or 24 weeks). In some examples, the severe psoriasis improves to mild, almost clear or no psoriasis (psoriasis of categories 0 to 2 according to the PtGA scale) during the treatment period.

### Method 8b

In other embodiments, the present disclosure provides a method of treating moderate psoriasis (according to either the Physician Global Assessment (PGA) scale or the Patient Global Assessment (PtGA) scale). The method comprises administering to a subject in need thereof a therapeutically effective amount of VB-201, wherein the therapeutically effective amount is described herein (e.g., the therapeutically effective amount is from about 80 mg/day to about 160 mg/day) for a treatment period of at least about 8 weeks (e.g., 8 weeks, 12 weeks, 16 weeks, or 24 weeks). In some examples, the moderate psoriasis improves to mild, almost clear or no psoriasis (psoriasis of categories 0 to 2 according to the PGA or the PtGA scale) during the treatment period.

### PASI Score

In some examples according to any one of the embodiments described herein, the subject, prior to the administering the VB-201, has a PASI score of at least about 10 and not more than about 20 (moderate to severe psoriasis based on PASI score). In some examples according to any one of the embodiments described herein, the subject, prior to the administering the VB-201, has a PASI score of less than about 10. In some examples according to any one of the embodiments described herein, the subject, prior to the administering the VB-201, has a PASI score of less than about 14.3. In other examples according to any of the embodiments described herein, the subject, prior to the administering of VB-201, has a PASI score that is from about 10 to about 20. In another example according to any of the embodiments described herein, the subject, prior to the administering of VB-201, has a PASI score that is from about 11 to about 20, or from about 12 to about 20, or from about 13 to about 20, or from about 14 to about 20, or from about 15 to about 20, or from about 10 to about 19, or from about 11 to about 19, or from about 12 to about 19, or from about 13 to about 19, or from about 14 to about 19, or from about 10 to about 18, or from about 11 to about 18, or from about 12 to about 18, or from about 13 to about 18, or from about 14 to about 18, or about 15 to about 18. In other examples according to any one of the embodiments described herein, the subject, prior to the administering of VB-201, has a PASI score that is from about 14.3 to about 18.5. In other examples according to any of the embodiments described herein, the subject, prior to the administering of VB-201, has a PAST score of greater than about 18.5.

### Method 9a

The present disclosure further provides a method of treating psoriasis, the method comprising administering to a subject in need thereof a therapeutically effective amount of VB-201, wherein the subject prior to the administering the VB-201 has a PASI score that is below 14.3, e.g., from about 10 to about 14 (e.g., from about 10 to about 13, or from about 10 to about 12, or from about 10 to about 11).

### Method 9b

The present disclosure further provides a method of treating psoriasis, the method comprising administering to a subject in need thereof a therapeutically effective amount of VB-201, wherein the subject prior to the administering the VB-201 has a PASI score that is from about 10 to about 20 (e.g., from about 14 to about 20, or from about 14 to about 19, or from about 14.3 to about 18.5) (e.g., moderate psoriasis). Other suitable ranges for the PASI score are disclosed herein. Preferably, the PASI score is from about 14 to about 20; more preferably, the PAST score is from about 14 to about 19; even more preferably, the PASI score is from about 14.3 to about 18.5.

### Method 9c

The present disclosure further provides a method of treating psoriasis, the method comprising administering to a subject in need thereof a therapeutically effective amount of VB-201, wherein the subject prior to the administering has a PASI score that is from about 18 to about 20 (e.g., from about 18.5 to about 20, or from about 19 to about 20).

The therapeutically effective amount according to Method 9a, 9b, or 9c is defined herein (e.g., the therapeutically effective amount is from about 80 mg/day to about 160 mg/day). In some preferred embodiments according to Method 9a, 9b, or 9c, the therapeutically effective amount is about 160 mg/day; preferably, the about 160 mg/day is administered in two daily sub-doses; more preferably, the about 160 mg/day is administered in two daily sub-doses of 80 mg administered about 12 hours apart. In some embodiments according to Method 9a, 9b, or 9c, the method comprising administering the VB-201 for a treatment period of at least about 8 weeks (e.g., 8 weeks, 12 weeks, 16 weeks, 20 weeks, or 24 weeks); preferably, at least about 12 weeks; more preferably, at least 24 weeks.

### Body Surface Area

In some examples according to any of the embodiments described herein, the subject, prior to the administering of the VB-201, has psoriasis (e.g., plaque psoriasis) characterized by (covering) a body surface area (BSA) of from about 10% to about 30%. In some examples according to any of the embodiments described herein, the subject, prior to the administering, has psoriasis characterized by a BSA of less than or equal to about 16%, e.g., from about 10% to about 16%. In other examples according to any of the embodiments described herein, the subject, prior to the administering, has psoriasis characterized by a BSA from about 10% to about 28%, or from about 10% to about 26%, or from about 10% to about 24%, or from about 12% to about 30%, or about 14% to about 30%, or about 16% to about 30%, or from about 12% to about 28%, or about 14% to about 28%, or about 16% to about 28%, or from about 12% to about 26%, or about 14% to about 26%, or about 16% to about 26%, or from about 12% to about 24%, or from about 14% to about 24%, or from about 16% to about 24%. In other examples according to any of the embodiments described herein, the subject, prior to the administering, has psoriasis characterized by a BSA of greater than or equal to about 24%, e.g., from about 24% to about 30%, from about 26% to about 30%, or from about 28% to about 30%.

### Method 10a

The present disclosure further provides a method of treating psoriasis, the method comprising administering to a subject in need thereof a therapeutically effective amount of VB-201, wherein the subject prior to the administering the VB-201 has a BSA of less than or equal to about 16% (e.g., from about 10% to about 16%).

### Method 10b

The present disclosure further provides a method of treating psoriasis, the method comprising administering to a subject in need thereof a therapeutically effective amount of VB-201 wherein the subject prior to the administering the VB-201 has a BSA from about 10% to about 30% (e.g., from about 14% to about 26%, or from about 16% to about 24%). Other ranges for BSA are disclosed herein. Preferably, the BSA is from about 14% to about 26%; more preferably, the BSA is from about 16% to about 24%.

### Method 10c

The present disclosure further provides a method of treating psoriasis, the method comprising administering to a subject in need thereof a therapeutically effective amount of VB-201, wherein the subject prior to the administering of the VB-201 has a BSA of greater than about 24%.

The therapeutically effective amount according to Method 10a, 10b, or 10c is defined herein (e.g., the therapeutically effective amount is from about 80 mg/day to about 160 mg/day). In some preferred embodiments according to Method 10a, 10b, or 10c, the therapeutically effective amount is about 160 mg/day; preferably, the about 160 mg/day is administered in two daily sub-doses; more preferably, the about 160 mg/day is administered in two daily sub-doses of 80 mg administered about 12 hours apart. In some embodiments according to Method 10a, 10b, or 10c, the method comprising administering the VB-201 for a treatment period of at least about 8 weeks (e.g., 8 weeks, 12 weeks, 16 weeks, 20 weeks, or 24 weeks); preferably, at least about 12 weeks; more preferably, at least 24 weeks.

Prior Treatment with Biologics or Immunosuppressants

In some examples according to any of the embodiments described herein, the subject, prior to the administering, has not been treated with a biologic psoriasis treatment. The term biologic, biologics, or biologic psoriasis treatment, or anti-psoriatic biologic means any biologic drug useful for the treatment of inflammation and/or autoimmune diseases, e.g., any form of psoriasis. Such biologics include, e.g., alefacept, which blocks molecules that dendritic cells use to communicate with T cells and causes natural killer cells to kill T cells as a way of controlling inflammation. Several monoclonal antibodies (MAbs) target inflammatory cytokines. TNF-α is one of the main executor inflammatory cytokines. Four MAbs (infliximab, adalimumab, golimumab and certolizumab pegol) and one recombinant TNF-α decoy receptor, etanercept have been developed against TNF-α to inhibit TNF-α signaling. Additional monoclonal antibodies have been developed against pro-inflammatory cytokines IL-12/IL-23 and Interleukin-17. The biologic drug adalimumab (Humira) was approved to treat moderate to severe psoriasis. Another biologic that has been approved for the treatment of moderate to severe psoriasis is ustekinumab (Stelara), an IL-12/IL-23 blocker.

### Method 11

The present disclosure further provides a method of treating psoriasis, the method comprising administering to a subject in need thereof a therapeutically effective amount of VB-201, wherein the subject was not treated with an anti-psoriatic biologic (e.g., did not undergo psoriasis treatment with a biologic) prior to first administering the VB-201 (e.g., during any time period prior to first administering the VB-201, or during a minimum time period immediately prior to first administering the VB-201). For example, the subject did not undergo psoriasis treatment with a biologic for at least about 2 months, at least about 4 months, at least, about 6 months, at least about 8 months, at least about 10 months, at least about 12 months, at least about 18 months, at least about 24 months, or at least about 32 months prior to first administering the VB-201). In another example, the subject has never received anti-psoriatic biologic treatment prior to first administering the VB-201.

In other examples according to any of the embodiments described herein, the subject, prior to the administering, has not been treated with an immunosuppressant drug. The term “immunosuppressant” includes all drug molecules known to lessen the immune reaction in a subject (e.g., a human subject), e.g., drugs useful to treat auto-immune diseases, such as psoriasis. The term immunosuppressant includes biologics, such as immunosuppressant antibodies, as well as non-biologic immunosuppressants. Exemplary non-biologic “immunosuppressants” include antimetabolites, such as folic acid analogues (e.g., methotrexate); purine analogues (e.g., azathioprine and mercaptopurine); pyrimidine analogues, protein synthesis inhibitors, cytotoxic antibiotics (e.g., dactinomycin, anthracyclines, mitomycin C, bleomycin, and mithramycin); calcineurin inhibitors (CNI) (e.g., cyclosporin, myriocin, tacrolimus, sirolimus), mycophenolate, and fingolimod.

### Method 12

Thus, the present disclosure further provides a method of treating psoriasis, the method comprising administering to a subject in need thereof a therapeutically effective amount of VB-201, wherein the subject was not treated with an immunosuppressant drug prior to first administering the VB-201 (e.g., during any time period prior to first administering the VB-201, or was not treated for at least a minimum time period prior to first administering the VB-201). For example, the subject did not undergo psoriasis treatment with an immunosuppressant for at least about 2 months, at least about 4 months, at least about 6 months, at least about 8 months, at least about 10 months, at least about 12 months, at least about 18 months, at least about 24 months, or at least about 32 months prior to first administering the VB-201). In another example, the subject has never received immunosuppressant treatment (e.g., for psoriasis or another reason) prior to first administering the VB 201.

Treatment Period

In some examples according to any one of the embodiments described herein (e.g., any one of the embodiments of Methods 6 to 12 described herein), the treatment period is at least about 12 weeks. In other examples according to any one of the embodiments described herein, the treatment period is at least about 16 weeks. In other examples according to any of the embodiments described herein, the treatment period is at least about 24 weeks. In yet other examples according to any of the embodiments described herein, the subject is treated with the VB-201 for a treatment period between about 12 weeks and about 24 weeks. In some examples according to any of the embodiments described herein, the VB-201 is administered to the subject as a short-term treatment (e.g., less than 3 months, less than 2 months, less than 1 months, less than 2 weeks, less than 1 week). In yet other examples according to any of the embodiments described herein, the VB-201 is administered to the subject as a long-term treatment (e.g., more than 1 year, more than 2 years, more than 5 years, or lifetime).

The length of treatment period may vary according to different doses. In some embodiments, the treatment period is at least about 12 weeks, wherein the daily dosage of VB-201 is about 80 mg. In some embodiments, the treatment period is at least about 16 weeks, wherein the daily dosage of VB-201 is about 80 mg. In some embodiments, the treatment period is at least about 24 weeks, wherein the daily dosage of VB-201 is about 80 mg. In some embodiments, the treatment period is at least about 8 weeks, at least about 12 weeks, at least about 16 weeks, or at least about 24 weeks, wherein the daily dosage of VB-201 is about 120 mg, or about 160 mg.

In some examples according to any one of the embodiments described herein (e.g., any one of the embodiments of Methods 6 to 12 described herein), the psoriasis is moderate to severe, stable, active plaque psoriasis vulgaris (psoriasis). In some examples, the moderate to severe, stable, active plaque psoriasis affects between about 10% to about 30% of the body surface of the subject and is characterized by a Psoriasis Area and Severity Index (PASI) score from about 10 to about 20.

In other examples according to any of the embodiments described herein (e.g., any of the embodiments of Methods 6 to 12 described herein), the subject prior to the administrating the VB-201 is characterized by having a PASI score of about 10 to about 20 (e.g., from about 14 to about 20, or from about 14 to about 19, or from about 14.3 to about 18.5); and a BSA of about 10% to about 30% (e.g., from about 14% to about 26%, or from about 16% to about 24%). In some examples, the subject prior to the administrating the VB-201 is characterized by having a PASI score of from about 14 to about 20 and a BSA of about 10% to 30%. In some examples, the subject prior to the administrating the VB-201 is characterized by having a PASI score of from about 14 to about 19 and a BSA of about 10% to about 30%. In some examples, the subject prior to the administrating the VB-201 is characterized by having a PASI score of from about 14.3 to about 18.5 and a BSA of about 10% to about 30%. In some examples, the subject prior to the administrating the VB-201 is characterized by having a PASI score of from about 14.3 to about 18.5 and a BSA of about 14% to about 26%. In some examples, the subject prior to the administrating the VB-201 is characterized by having a PASI score of from about 10 to about 20 and a BSA of about 14% to about 26%. In some examples, the subject prior to the administrating the VB-201 is characterized by having a PASI score of from about 10 to about 20 and a BSA of about 16% to about 24%. In some examples, the subject prior to the administrating the VB-201 is characterized by having a PASI score of from about 14.3 to about 18.5 and a BSA of about 14% to about 26%. In some examples, the subject prior to the administrating the VB-201 is characterized by having a PASI score of any of the suitable ranges described herein and a BSA of any of the suitable ranges described herein. In any of the above examples, the subject is further characterized by that prior to first administrating the VB-201, the subject was not treated with an anti-psoriatic biologic or an immunosuppressant as described herein.

In other examples according to any of the embodiments described herein (e.g., any of the embodiments of Methods 6 to 12 described herein), the subject has a diagnosis of chronic plaque psoriasis for at least about 6 months prior to administering the VB-201 to the subject.

In some examples according to any of the embodiments described herein (e.g., any of the embodiments of Methods 6 to 12 described herein), the psoriasis is plaque psoriasis.

### Formulations

In any of the embodiments described herein (e.g., any of the embodiments of Methods 1a to 12 described herein), the VB-201 administered to the subject may be formulated as a pharmaceutical composition for any suitable routes of administration, for example, oral, rectal, topical, transdermal, transmucosal (especially transnasal), intestinal or parenteral delivery, including intramuscular, subcutaneous and intramedullary injections as well as intrathecal, direct intraventricular, intravenous, intraperitoneal, intranasal, or intraocular injections.

For preparing pharmaceutical compositions comprising VB-201, inert, pharmaceutically acceptable carriers can be either solid or liquid. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

For oral administration, the pharmaceutical composition can be formulated readily by combining VB-201 with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, VB-201 may be delivered in the form of an aerosol spray presentation from a pressurized pack or a nebulizer with the use of a suitable propellant, e.g., dichlorodifluoromethane, trichlorofluoromethane, dichloro tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of VB-201 and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

VB-201 may be administered topically or transdermally. Thus, VB-201 may be formulated as a topical or transdermal compositions. The topical or transdermal compositions can take the form of creams, gels, foams, lotions, aerosols, ointments, and/or emulsions and can be included in a transdermal patch of the matrix or reservoir type as are conventional in the art for this purpose.

Preferably VB-201 is administered orally.

In some examples according to any one of the embodiments of Methods 1a to 12 described herein, the VB-201 administered to the subject is formulated for oral administration, e.g., in a liquid-fill hard-gelatin capsule. Exemplary VB-201 formulations useful in the context of this disclosure are described in PCT/US2012/053533 to Sher et. al., the disclosure of which is incorporated herein by reference in its entirety.

In some examples according to any of the embodiments described herein, the VB-201 is formulated using a thermosoftening carrier selected from a poloxamer (e.g., poloxamer 188) and a polyethylene glycol having a molecular weight from about 6000 to about 8000 (e.g., PEG6000), an anti-adherent agent (e.g., talc) at a weight ratio from about 1:4 to about 1:1 (anti-adherent agent:VB-201), and a thixotropic agent (e.g., fumed silicon dioxide) at a concentration relative to the combined weight of the thermosoftening carrier and the thixotropic agent from about 0.5 weight percent to about 5 weight percent (e.g., from about 1 weight percent to about 3 weight percent). In some examples according to any one of the embodiments of Methods 1a-12 described herein, the VB-201 is administered to the subject using a formulation comprising a poloxamer (e.g., polaxamer 188) as a thermosoftening carrier, VB-201 from about 20 mg to about 80 mg, talc at a weight ratio of about 1:1 or at a weight ratio of about 1:4 (talc:VB-201), and fumed silicon dioxide as a thixotropic agent at a concentration relative to the combined weight of the poloxamer and the fumed silicon dioxide from about 1 weight percent to about 3 weight percent.

In some examples according to any one of the embodiments described herein, the VB-201 is administered to the subject in a formulation comprising: 40 mg VB-201, 40 mg of an anti-adherent agent (e.g., talc), 12 mg of a thixotropic agent (e.g., fumed silica), and 388 mg of a thermosoftening carrier (e.g., a poloxamer). In other examples, the VB-201 is administered to the subject in a formulation comprising: 40 mg VB-201, 40 mg talc, 12 mg of fumed silicon dioxide, and 388 mg of a poloxamer. In other examples, the VB-201 is administered to the subject in a formulation comprising: 40 mg VB-201, 10 mg of an anti-adherent agent (e.g., talc), 4 mg of a thixotropic agent (e.g., fumed silica), and 396 ma of a thermosoftening carrier (e.g., a poloxamer). In other examples, the VB-201 is administered to the subject in a formulation comprising: 40 mg VB-201, 10 mg talc, 4 mg fumed silicon dioxide, and 396 mg of poloxamer 188. In other examples, the VB-201 is administered to the subject in a formulation comprising: 80 mg VB-201, 80 mg of an anti-adherent agent (e.g., talc), 12 mg of a thixotropic agent (e.g., fumed silica), and 388 mg of a thermosoftening carrier (e.g., a poloxamer). In other examples, the VB-201 is administered to the subject in a formulation comprising: 80 mg VB-201, 80 mg talc, 1.2 mg fumed silica, and 388 mg of poloxamer 188. In other examples, the VB-201 is administered to the subject in a formulation comprising: 80 mg VB-201, 20 mg of an anti-adherent agent (e.g., talc), 4 mg of a thixotropic agent (e.g., fumed silica), and 396 mg of a thermosoftening agent. In other examples, the VB-201 is administered to the subject in a formulation comprising: 80 mg VB-201, 20 mg talc, 4 mg fumed silicon dioxide, and 396 mg of poloxamer 188.

### Thermosoftening Carrier

As used herein, the term “thermosoftening carrier” refers to a carrier which becomes soft (e.g., a fluid) upon heating to a temperature above room temperature. A thermosoftening carrier becomes soft at a temperature which does not damage the active pharmaceutical ingredient (e.g., by oxidation) or the thermosoftening carrier itself. The softening upon heating may be either characterized by a phase transition (e.g., a solid-to-liquid transition), or not characterized by a phase transition (e.g., softening of an amorphous material). The thermosoftening is reversible, such that the softened carrier becomes harder upon being cooled back to room temperature. In some embodiments, the thermosoftening carrier is a mixture of two or more agents.

The thermosoftening carrier facilitates preparation of a liquid fill composition and filling of capsules therewith at a temperature at which the thermosoftening carrier is soft, as well as formation of a solid or semi-solid matrix following cooling (e.g., cooling to room temperature). In one example, the thermosoftening carrier is a solid or a semi-solid at a temperature below 35° C., or below 30° C. (e.g., at room temperature, i.e., 25° C.). In one example, the thermosoftening carrier is non-hygroscopic. The thermosoftening carrier is a pharmaceutically acceptable carrier.

Optionally, the thermosoftening carrier becomes soft at a temperature of no more than about 150° C., and optionally at a temperature of no more than about 100° C., or 90° C.

In some embodiments, the thermosoftening carrier has a melting point in a range of from about 40° C. to about 100° C. Optionally, the melting point is in a range of from about 50° C. to about 80° C. In other examples, the melting point of the thermosoftening carrier is from about 50° C. to about 70° C., or from about 50° C. to about 60° C., and optionally in a range of from about 55° C. to about 65° C. Accordingly, at such temperatures, the thermosoftening carrier undergoes transformation from a hard to a soft material, and vice versa. In one example, the thermosoftening carrier at a temperature above its melting point is sufficiently soft for filling the carrier into a capsule (e.g., into a hard gelatin capsule).

Examples of thermosoftening carriers include waxes, poloxamers (e.g., Poloxamer 188), macrogol glycerides, high-molecular weight PEGs (e.g., PEG6000 or PEG 8000), glycerol monooleates or monostearates, hydrogenated or partially hydrogenated glycerides (e.g., hydrogenated palm kernel oil or hydrogenated cotton seed oil)), Gelucires™, and hard fats such as beeswax. Other exemplary thermosoftening carriers include Softisan™ and hexadecane-1-ol.

In some embodiments, the polyalkylene glycol is a poloxamer. Accordingly, in some embodiments, the thermosoftening carrier is a poloxamer.

Poloxamers are triblock polyalkylene glycols, comprising a central polypropylene glycol chain, which is relatively hydrophobic, flanked by two polyethylene glycol chains, which are relatively hydrophilic. This combination of hydrophobic and hydrophilic chains provides poloxamers with surfactant properties.

Poloxamers are typically characterized by molecular weight of the polypropylene glycol core of the poloxamer and by the proportion of polyethylene glycol versus polypropylene glycol. These parameters are commonly described by character′zing a poloxamer with a three-digit number, wherein the first two digits, when multiplied by 100, give the molecular weight (in daltons) of the polypropylene glycol core, whereas the last digit, when multiplied by 10, gives the percentage of polyethylene glycol. Thus, for example, poloxamer 188 has a polypropylene glycol core with a molecular weight of 1800 daltons and is 80% polyethylene glycol (and thus has a total molecular weight of approximately 9000 daltons), whereas poloxamer 407 has a polypropylene glycol core with a molecular weight of 4000 daltons and is 70% polyethylene glycol (and thus has a total molecular weight of approximately 13000 daltons).

Poloxamer 188 is an exemplary poloxamer. Accordingly, in some embodiments, the thermosoftening carrier is poloxamer 188.

In one embodiment, the thermosoftening carrier is selected from PEG6000, poloxamer 188, and combinations thereof.

The thermosoftening carrier may also comprise an oil or a combination of one or more oils. Many oils suitable for use as a thermosoftening carrier for therapeutic applications are known in the art. Examples include, without limitation, esters of fatty acids, such as triglycerides and diesters of a glycol (e.g., propylene glycol). Other oils may be added to the thermosoftening carrier to decrease/fine tune viscosity, e.g., fractioned coconut oil or soybean oil.

### Anti-Adherent Agent

As used herein, the phrase “anti-adherent agent” refers to an agent which reduces the cohesion between particles of a substance (e.g., VB-201) and/or an adherence of such particles to a solid surface (e.g., of a container and/or encapsulation machinery). For example, the reduction of cohesion caused by an anti-adherent agent is greater than a reduction of cohesion caused by mere dilution of the substance by addition of an agent.

Optionally, the anti-adherent agent is a material (e.g., a solid, such as a powder) with little or no solubility in the other components of the capsule (e.g., the thermosoftening carrier). The anti-adherent agent may act by adhering to the VB-201 thereby forming, e.g., grains and/or powder particles. As a result, the adherence of the VB-201 to other surfaces (e.g., other VB-201 grains and/or powder particles, surfaces of containers and/or encapsulation machinery) is reduced.

In some embodiments, the anti-adherent agent to VB-201 ratio is about 1:10 to about 10:1, about 1:8 to about 8:1, about 1:5 to about 5:1, about 1:4 to about 4:1; about 1:4 to about 3:1; about 1:4 to about 2:1, or about 1:4 to about 1:1. In exemplary embodiments, the anti-adherent agent to VB-201 ratio is about 1:1, about 5:1, about 1:5, or about 1:4. In some embodiments, the pharmaceutical composition has a concentration of the anti-adherent agent of about 1% to about 45% by weight of total weight of the pharmaceutical composition. In some embodiments, the pharmaceutical composition has a concentration of the anti-adherent agent of about 1% to about 30% by weight of total weight of the pharmaceutical composition. In some embodiments, the pharmaceutical composition has a concentration of the anti-adherent agent of about 30% to about 45%, about 20% to about 30%, about 10% to about 20%, about 1% to about 20%, about 1% to about 15%, or about 1% to about 10% by weight of total weight of the pharmaceutical composition. In some embodiments, the pharmaceutical composition has a concentration of the anti-adherent agent of about 30%, about 40%, about 45%, about 25%, about 20%, about 15%, about 10%, about 5%, about 2%, about 1% by weight of total weight of the pharmaceutical composition. In some embodiments, the pharmaceutical composition has a concentration of the anti-adherent agent of about 31%, about 39%, about 45%, about 8%, about 3.1%, about 2.2%, about 2.5%, about 3.2%, about 1.8% by weight of total weight of the pharmaceutical composition. In some embodiments, the pharmaceutical composition has a concentration of the anti-adherent agent of more than about 30%, more than about 45% by weight of total weight of the pharmaceutical composition. In any of the embodiments described herein, the anti-adherent agent can be talc.

Examples of anti-adherent agents include, but are not limited to, talc, magnesium stearate, cellulose (e.g., microcrystalline cellulose), cellulose derivatives (e.g., hydroxypropyl methylcellulose (HPMC)), lactose, gelatin, alginates, aluminium hydroxide, magnesium oxide, clays, attapulgite, bentonite, carrageenan, copovidone, hectorite, polymethacrylates, sodium docusate, erythritol, povidones, croscarmellose sodium, dextrates, starches, iron oxide, kaolin, silicates (e.g., magnesium aluminium silicate), corn flour, sugars, calcium carbonate, magnesium carbonate, calcium phosphate, calcium sulfate, bicarbonates (e.g., of potassium or sodium), citrate salts (e.g., potassium citrate) and titanium dioxide.

In one example according to any of the embodiments described herein, the anti-adherent agent is talc. Any pharmaceutical-grade or food-grade talc (e.g., powdered talc) may be used. Exemplary grades of talc, which can be used in the pharmaceutical compositions, liquid-fill compositions, capsules and other are embodiments herein are disclosed in Dawoodbhai et al., “Pharmaceutical and Cosmetic Uses of Talc,” Drug Development and Industrial Pharmacy, 16(16):2409-2429 (1990); and Dawoodbhai et al., “Glidants and Lubricant Properties of Several Types of Talcs,” Drug Development and Industrial Pharmacy, 13(13):2441-2467 (1987), each of which is incorporated herein by reference in its entirety. In some examples, the talc is powdered talc. In some examples, the talc is of USP grade. In other example, the talc is powdered talc and of USP grade.

### Thixotropic Agent

As used herein, a “thixotropic agent” refers to an agent which increases a viscosity of a liquid when added to a liquid. As known in the art “thixotropy” is a reversible behaviour of viscous liquids (e.g., gels) that liquefy when subjected to shear stress such as shaking or stirring, or otherwise disturbed.

A viscous liquid containing a thixotropic agent exhibits thixotropy, wherein the viscosity is reduced under stress (e.g., stirring, heating and/or application of shear forces). The ingredients in a liquid fill composition (e.g., carrier, VB-201, thixotropic agent, and/or anti-adherent agent) can therefore be readily mixed by stirring, as the viscosity is reduced during stirring, yet the fill composition is relatively resistant to separation of components, as the viscosity increases when stirring ceases.

Examples of thixotropic agents suitable for use in the context of the present embodiments include, but are not limited to fumed silica (available, for example as Aerosils® and Cab-O-Sil® products), kieselguhr, gums (e.g., xanthan gum, guar gum, locust bean gum, alginates), cellulose derivatives (e.g., hydroxypropyl methyl cellulose), starches, polymers (e.g., polyvinyl alcohol, polyacrylates, hydrophobically-modified polyacrylates), emulsifiers, and clay derivatives (e.g., amine treated magnesium aluminum silicate, bentonite colloidal silicic acid, white smectite clays and bleaching earth, attapulgite, mica, synthetic magnesium phyllosilicates (Laponite), layered silicates, modified smectites, hectorite, and sepiolite. Optionally, the thixotropic agent comprises fumed silica and/or attapulgite.

The concentration of the thixotropic agent in the pharmaceutical composition (i.e., liquid-fill composition or matrix of the capsule) unless otherwise indicated is determined relative to the combined weight of the thermosoftening carrier and the thixotropic agent. For example, at 2.5 weight percent of thixotropic agent, the pharmaceutical composition may contain 10 mg thixotropic agent and 390 mg of a thermosoftening carrier (10/400=2.5%).

In one example according to any of the embodiments described herein, the thixotropic agent is a different substance than the thermosoftening agent (i.e., the thixotropic agent is chemically distinct from the thermosoftening agent). In other examples according to any of the embodiments described herein, the thixotropic agent is a different substance than the anti-adherent agent (i.e., the thixotropic agent is chemically distinct from the anti-adherent agent). In other examples according to any of the embodiments described herein, the thixotropic agent is a different substance than the thermosoftening agent and the anti-adherent agent (i.e., the thixotropic agent is chemically distinct from both the thermosoftening agent and the anti-adherent agent).

In various embodiments, the present disclosure also provides for a pharmaceutical composition comprising VB-201. In some embodiments, the pharmaceutical composition can be any pharmaceutical composition described herein.

Exemplary pharmaceutical compositions of the present disclosure are shown below. In any of the embodiments disclosed herein, the pharmaceutical composition comprising VB-201 can be any of the exemplary pharmaceutical compositions described below.

Exemplary Pharmaceutical Compositions with Talc to VB-201 Ratio of 1:4

| | | 60 mg capsules | 80 mg capsules |
|---|---|---|---|
| Dose | 40 mg capsules (mg) | (mg) | (mg) |
| VB-201 | 40 | 60 | 80 |
| Aerosil (1%) | 4 | 4 | 4 |
| poloxamer 188 | 396 | 396 | 396 |
| Talc(1:4) | 10 | 15 | 20 |
| Talc % weight | 10/450 = 2.2% | 15/475 = 3.1% | 20/500 = 8% |

Exemplary Pharmaceutical Compositions with Talc to VB-201 Ratio of 5:1

| | 40 mg capsules | 60 mg capsules | 80 mg capsules |
|---|---|---|---|
| Dose | (mg) | (mg) | (mg) |
| VB-201 | 40 | 60 | 80 |
| Aerosil (1%) | 4 | 4 | 4 |
| poloxamer 188 | 396 | 396 | 396 |
| Talc (5:1) | 200 | 300 | 400 |
| Talc % weight | 200/640 = 31% | 300/760 = 39% | 400/880 = 45% |

Exemplary Pharmaceutical Compositions with Talc to VB-201 Ratio of 1:5

| | 40 mg capsules | 60 mg capsules | 80 mg capsules |
|---|---|---|---|
| Dose | (mg) | (mg) | (mg) |
| VB-201 | 40 | 60 | 80 |
| Aerosil (1%) | 4 | 4 | 4 |
| poloxamer 188 | 396 | 396 | 396 |
| Talc(1:5) | 8 | 12 | 16 |
| Talc % | 8/448 = 1.8% | 12/472 = 2.5% | 16/496 = 3.2% |
| weight % | | | |

### Combined Treatment of Psoriasis

VB-201 in the present disclosure may be used as a stand-alone therapy for treating psoriasis, or as an adjunct therapy to be combined with one or more other treatments of psoriasis.

The other treatments for psoriasis are known in the art, including topical treatments, systemic treatments, and photo treatments. Topical agents useful for treating psoriasis are known in the art; non-limiting examples include corticosteroids (e.g., desonide, flumethasone pivalate, fluocinolone acetonide, hydrocortisone, amcinonide, clocortolone, desoximetasone, betamethasone, etc.), Vitamin D-3 related drugs (e.g., calcipotriene, maxacalcitol, tacalcitol, calcitriol, etc.), coal tar, anthralin, calcineurin inhibitors (e.g., tacrolimus, pimecrolimus), retinoids (e.g., tazarotene), and salicyclic acid. Systemic agents useful for treating psoriasis are also known in the art; non-limiting examples include small molecule drugs, such as cyclosporine, methotrexate, and retinoids, and biologics. Non-limiting, exemplary biologics that have been proven to be useful in treating psoriasis include T-cell blockers (e.g., alefacept), TNF blockers (e.g., etanercept, infliximab, adalimimab), and IL-12 and/or IL-23 blockers (e.g., ustekinumab). In addition, phototherapy (i.e., photo treatment, treat with light, e.g., UV light therapy) may also be employed for treating certain patients having psoriasis.

Thus, the present disclosure further provides a method of combined treatment of psoriasis. Any of the above topical treatments, systemic treatments, photo treatment, or any combinations thereof may be combined with any of the above methods of treating psoriasis in a subject in need thereof comprising administering to the subject a therapeutically effective amount of VB-201 In some embodiments, the combined treatment comprising administering to the subject a therapeutically effective amount of an additional therapeutic agent, wherein the additional therapeutic agent is a topical agent useful for treating psoriasis. Suitable topical agents useful for treating psoriasis are known in the art, for example, as described herein. In other embodiments, the combined treatment comprising administering to the subject a therapeutically effective amount of an additional therapeutic agent, wherein the additional therapeutic agent is a systemic agent useful for treating psoriasis. Suitable systemic agents useful for treating psoriasis are known in the art, for example, as described herein. In yet other embodiments, the combined treatment comprising treating the subject with a suitable photo treatment. Suitable photo treatments are known in the art, for example, treating psoriatic patients with ultraviolet (UV) light, e.g., UV A or UV B.

In some embodiments, the combined treatment is for treating moderate psoriasis, moderate to severe, severe, or worst psoriasis has ever been in a subject. In some preferred embodiments, the combined treatment is for treating moderate psoriasis. In some more preferred embodiments, the combined treatment is for treating severe psoriasis or worse than severe psoriasis.

In some examples according to any one of the embodiments described herein, the subject is a human patient.

In some examples according to any of the embodiments described herein, the VB-201 is administered with food.

### EXAMPLES

### Example 1

### Safety and Efficacy of VB-201 on Vascular Inflammation of Atherosclerosis as Measured by FDG PET/CT Imaging

The safety and efficacy of VB-201 was evaluated for treatment of patients with moderate to severe plaque psoriasis. As psoriasis is associated with increased atherosclerotic cardiovascular morbidity and mortality, VB-201's effect on vascular inflammation using 18-fluorodeoxyglueose (18-FDG) PET/CT imaging of the carotid arteries and ascending aorta was evaluated.

### Methods:

### Overview of the Phase 2 Clinical Trial Design

The general study design for the phase 2 clinical trial is shown in FIG. 2. Patients were screened for eligibility and, up to 28 days later, at the baseline visit, randomized to one of three treatment groups (1:1:1): VB-201 20 mg/day: VB-201 80 mg/day: placebo/day. Patients consumed 2 capsules per day (VB-201 20 mg+placebo, VB-201 40 mg+40 mg, or 2× placebo). To ensure that the treatment assignments were concealed, all patients received 2 matching capsules of VB-201 or placebo.

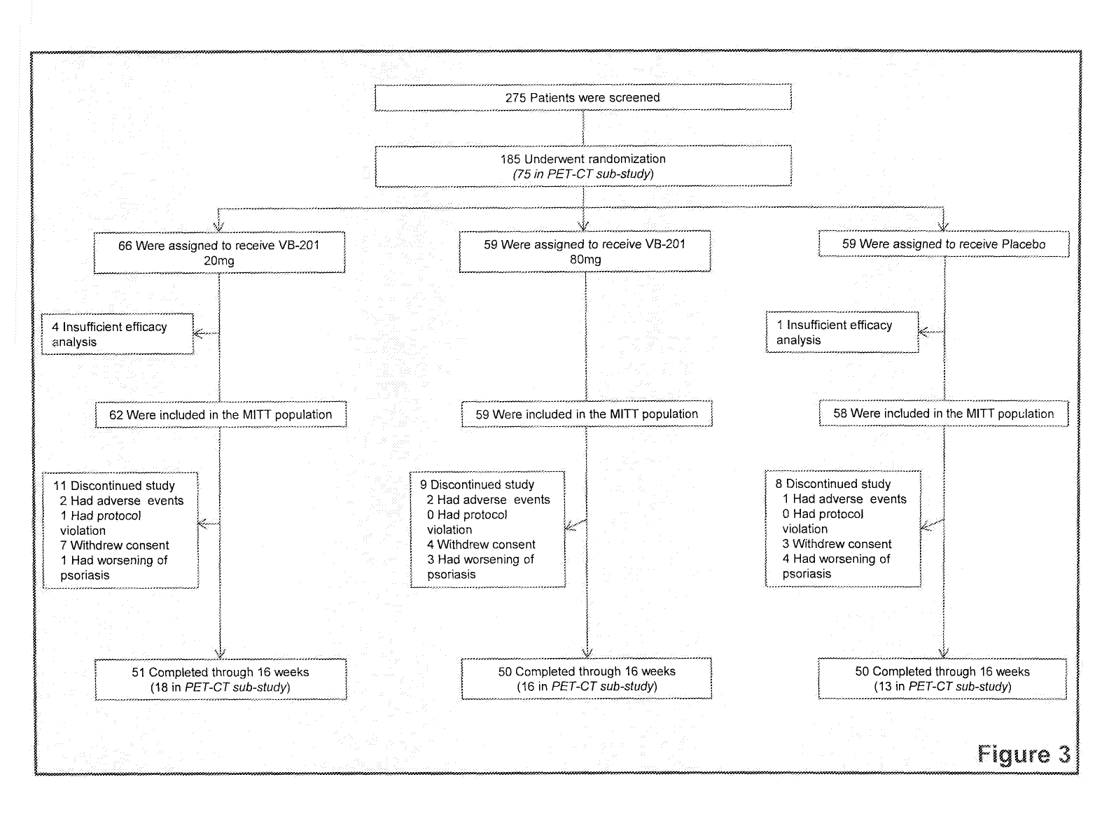

FIG. 3 shows a schematic view of the screening, randomization, and follow-up of the Phase 2 clinical trial.

The efficacy analyses were performed on the data from a modified intention-to-treat (MITT) population (defined as all patients randomized who received at least one dose of medication and had at least one efficacy evaluation). The safety analyses were performed on all randomized patients who received at least one dose of the drug.

### The PET-CT Sub-Study

This study was a sub-study of the above phase 2, randomized, double-blind, multicenter trial, of patients (aged 18-75) with moderate to severe plaque psoriasis, see also infra at Example 3. Patients (Table 1, below) had an increased cardiovascular risk and increased vascular inflammation in the ascending aorta, right or left carotid artery, defined as a target background ratio (TBR) of at least 1.6 on a baseline 18-FDG PET/CT. In addition, patients enrolled into the sub-study had to have the presence or history of one of the following: non-insulin dependent diabetes mellitus (NIDDM), peripheral vascular disease (PVD), smoker, hypertension, ischemic heart disease or cerebrovascular disease; obesity (BMI ≧25), hypercholesterolemia (LDL>160 mg/dL or taking anti-hyperlipidemia treatment), or family history of ischemic heart disease (in brother or father before age 55, in sister or mother before age 65) or at least 40 years of age with a history of psoriasis for at least 7 years. Patients were randomly assigned (1:1:1) to receive VB-201 20 mg, VB-201 80 mg or placebo for 12 weeks. The primary endpoint was the maximum TBR in the most diseased segment (MDS) of an index vessel at 12 weeks (percent change from baseline). The effect of treatment with 20 mg per day and 80 mg per day VB-201 on carotid arteries and ascending aorta inflammation was examined. Inflammation was measured by positron emission computed tomography (PET-CT) to quantify 18-fluorodeoxyglucose (18-FDG) uptake as a target to background ratio (TBR).

18-MG-PET imaging was performed following an overnight fast using a PET-CT scanner system with CT attenuation correction. 18-FDG was administered intravenously at a dose of 370 MBq and PET images were acquired approximately 90 minutes after injection. Baseline scans were evaluated and all inflammatory plaques in the left and right carotid arteries and ascending aorta were identified and totaled for each treatment group. PET scans were performed at Harvard Medical School and Massachusetts General Hospital, Boston, Mass., USA and at Translational and Molecular Imaging Institute and Department of Radiology, Mount Sinai School of Medicine, New York, N.Y., USA. All scans were analyzed by a central reader at the Mount Sinai center in New York City. The TBR was calculated from the ratio of the standard uptake values (SUV) of the target compared to background venous activity:

TBR (Target Background Ratio)=Target activity SUVTarget/SUV Background Venous Activity

The baseline and 12-week PET/CT datasets for each patient were structurally co-registered with each other, and FDG uptake was compared across each patient's images within pre-defined sections of the target vessels. For the aorta, measurements were made every 3 mm, starting 1 cm above the aortic valve annulus, continuing to the bottom of the aortic arch. For the right and left carotids, measurements were made every 3 mm, starting at the level of the carotid artery bifurcation, and continuing inferiorly 4 cm into the common carotid artery. Measurements were made in the axial plane by drawing a region of interest (ROI) around the artery wall and the maximum standardized uptake value (SUV) was recorded as the time- and dose-corrected tissue radioactivity divided by body weight. This represented the values for the target tissue. For the carotid arteries, the SUV Mean from the lumen of the jugular vein was used in at least 10 consecutive slices (at least 5 on each side). For the aorta, the background SUVMean from the atrium or SVC was used in at least 10 consecutive slices.

All statistical tests were two-sided with a significance level of 5%, unless specified otherwise, and were performed using SASS Version 9.2. Data was summarized using descriptive statistics (number of patients [N], mean, standard deviation [SD], median, minimum, and maximum).

Subjects were scanned by PET-CT to determine baseline 18-FDG uptake, and those with a TBR of at least 1.6 in either the carotid artery or the ascending aorta were selected. After screening and establishment of a baseline, eligible subjects were randomly assigned to receive 20 mg per day VB-201, 80 mg per day VB-201 or a daily placebo, for a period of 12 weeks.

Doses were administered orally at breakfast time with food. The maximal dose of VB-201 (80 mg/day) was determined according to earlier tolerability results (see, e.g., Example 4 of WO2011/083465). The TBR of each subject was determined again by PET-CT at week 12 of the treatment. Efficacy was determined by comparing TBR values obtained before and after treatment.

The vessel with the highest FDG uptake at baseline was identified as the index vessel. Thereafter, the average of the maximum TBR activity within the most diseased segment (MDS) of the index vessel (MDS TBR) was recorded. The MDS is centered on the slice of artery demonstrating the highest FDG uptake at baseline, and includes the neighboring inferior and superior axial slices. The MDS TBR is calculated as a mean of maximum TBR values derived from those three contiguous axial segments.

### Results:

One hundred and eighty five patients were randomized to the main psoriasis study. Fifty eight (86%) of the 67 patients randomized to the sub-study had increased vascular inflammation at baseline, 47 completed 12 weeks of therapy and were evaluable for analysis. The mean age was 49 (range 23-68), 64% were male, 83% were obese and 28% were on concomitant statin therapy (Table 1, below). VB-201 was safe and well tolerated, and had no effect on lipid levels.

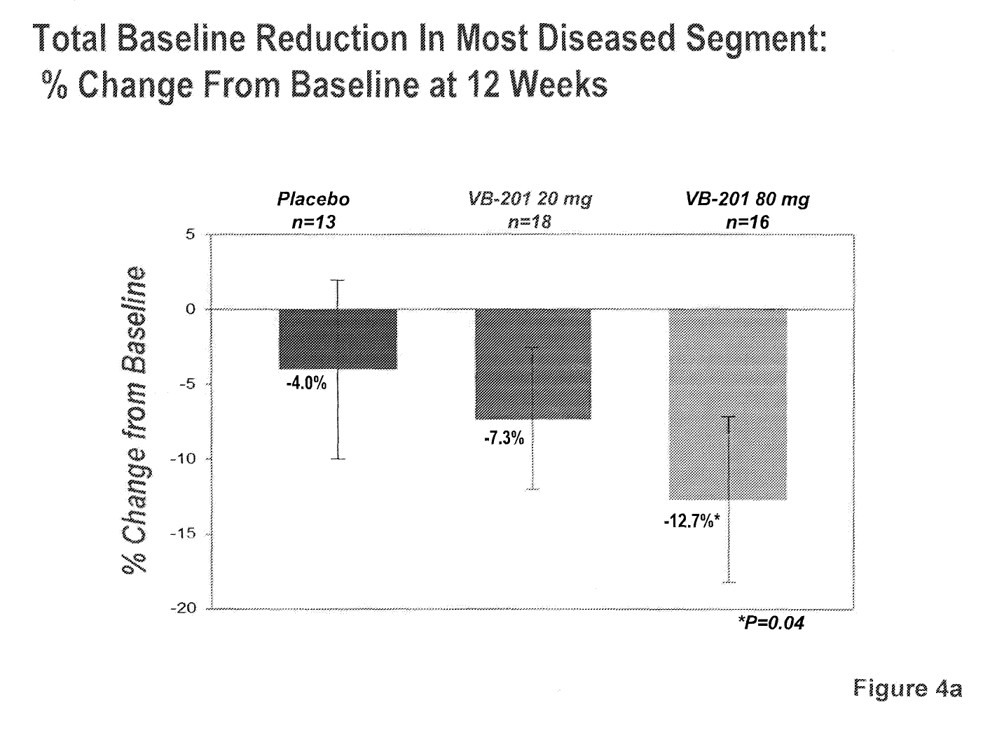

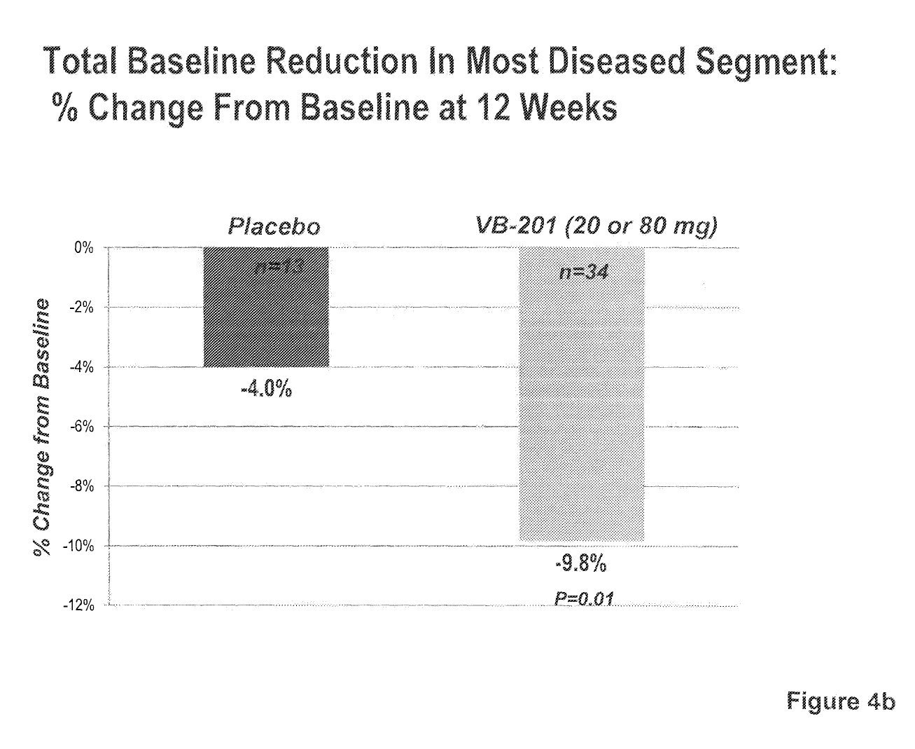

The study met the primary endpoint, see FIGS. 4A and 4B, and demonstrated a statistically significant reduction in vascular inflammation based on PET-CT measure (a 12.7% reduction from baseline in the VB-201 80 mg group (p=0.04), and a 9.8% reduction from baseline in combined VB-201 treated group (p=0.01). The change from baseline in 18-FDG-PET MDS TBR of the index vessel in patients treated with VB-201 was:

VB-201 20 mg/day: −7.3% [95%−7.32−2.70, p=0.14],

VB-201 80 mg/day: −12.7% [CI 95%−24.51%−−0.89%, p=0.04]

Placebo: −4.0% [CI 95%−17.02−9.01, p=0.52]

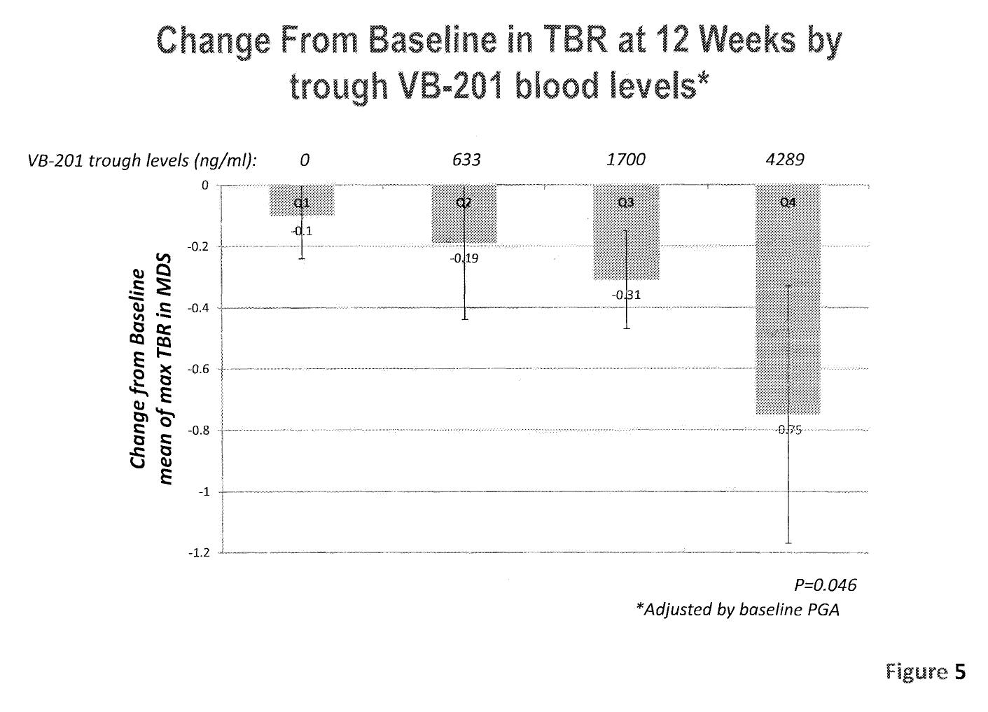

A dose response was seen across quartiles of VB-201 trough blood levels (p=0.037). See FIG. 5. A similar effect of VB-201 on TBR was seen in patients with or without statins.

There were 34/47 patients (72%) who were not taking concomitant statins (12 in the placebo group, 10 in the 20 mg group and 10 in the 80 mg group) and there were 13/47 patients (28%) who were taking concomitant statins (1 in the placebo group, and 6 in each of the 20 mg and 80 mg dose groups).

Among patients who were not taking any concomitant statin medications, a trend was seen in the mean change and percent change from baseline of the maximum TBR in the MDS in the index vessel of patients in the 80 mg group (−0.39 and −8.84% change from baseline and percent change, respectively).

Among patients who were taking statin medications while taking VB-201 (6 patients on VB-201 20 mg and 6 patients on VB-201 80 mg), the mean change and percent change from baseline of the maximum TBR in the MDS in the index vessel was −0.74 and −18.48% (p=0.01).

### Conclusion:

VB-201 has an anti-inflammatory effect in atherosclerosis of the vascular wall in human patients. This anti-inflammatory effect is also seen in patients undergoing statin therapy (e.g., patients who are treated with a statin prior to treatment with VB-201 and/or are concomitantly treated with a statin).

### Example 2

### Efficacy of VB-201 in a Patient with Inflammation Associated with an Implant

A patient with breast implants was treated with VD-201 at 20 mg/day for 12 weeks. PET-CT scans were done at baseline and at 12 weeks, using positron emission computed tomography (PET/CT) imaging quantifying 18-fluorodeoxyglucose (18-FDG) uptake as a TBR as described in Example 1.

The efficacy of VB-201 in treating inflammation associated with the breast implant is shown in FIG. 6. The patient had inflammation associated with the breast implants prior to the treatment of VB-201, see the white arrows pointing at 18-FDG uptake near the breast implants in FIG. 6. The patient had reactive inflammation in response to the implants, which may tissues surrounding the implants. At 12 weeks, reduced inflammation surrounding the breast implants of the patient was observed, see the white arrows pointing at 18-FDG uptake near the breast implants in FIG. 6. However, the effect of VB-201 in reducing inflammation associated with the breast implants might have occurred earlier than 12 weeks.

The efficacy of VD-201 in treating inflammation of the ascending aorta in the patient is also shown in FIG. 6. The patient had inflammation of an aorta prior to VB-201 treatment. The images in FIG. 6 show that upon VB-201 treatment, inflammation of the aorta in the patient was also reduced, see the black arrows pointing at 18-FDG uptake in the ascending aorta in FIG. 6.

### Example 3

### Efficacy of VB-201 in Patients with Plaque Psoriasis

A randomized, double-blind Phase II clinical study was performed in subjects with moderate to severe plaque psoriasis, in order to determine the efficacy of VB-201 in treating this condition. Patients (men or women in the age of 18-75 years) were selected if they had a diagnosis of plaque psoriasis for at least 6 months and they had moderate (i.e., scoring at least 3 on a 0 to 5 point Physician Global Assessment (PGA) scale) to severe, stable and active plaque psoriasis vulgaris affecting at least 10% of the body surface and with a Psoriasis Area and Severity Index (PASI) score of at least 12. Patients underwent a wash-out period following any previous treatments. After screening and establishment of a baseline, eligible subjects were randomly assigned to receive 20 mg per day VB-201, 80 mg per day VB-201 or a daily placebo, for a period of 12 weeks.

Doses were administered orally at breakfast time with food. The maximal dose of VB-201 (80 mg/day) was determined according to earlier tolerability results (see, e.g., Example 4 of WO2011/083465).

Efficacy of each dosage level of VB-201 relative to placebo was determined according to the percentage of patients which achieved at least a 50% or 75% improvement over baseline PASI score at week 12.

Additional criteria included change in affected body surface area from baseline to week 12, change in PGA score from baseline to week 12, any change relative to baseline PASI score, and change in Patient Psoriasis Global Assessment (PtGA) score from baseline to week 12. Blood samples were taken at weeks 4, 8 and 12, in order to assess VB-201 pharmacokinetics, as well as plasma cytokine levels. Safety of VB-201 administration was evaluated by physical examination, incidence of adverse effects, vital signs, clinical chemistry, hematology, urinalysis and electrocardiograms.

Statistical comparisons were performed using a two-sided comparison with a 5% level of significance. All analyses were performed using Statistical Analysis Software (SAS®) Version 9.2.

### Analysis of PGA and PtGA Scores

Categorical analyses of Physician's Global Assessment and Patient's Psoriasis Global Assessment scores at Baseline, Week 8 and Week 12 were analyzed.

The Physician's Global Assessment (PGA) score was based on a 5-point scale with possible outcomes 0=clear, 1=almost clear, 2=mild, 3=moderate and 4=severe.

The Patient's Psoriasis Global Assessment (PtGA) score was based on a 6-point scale with possible outcomes 0=clear, 1=almost clear, 2=mild, 3=moderate, and 4 severe and 5=worst psoriasis has ever been.

The analyses that follow use the categorical nature of the PGA and PtGA, i.e. the 5-point or 6-point ordered scale which was considered more relevant than a continuous approach.

The proportion of patients with baseline, week 8 and week 12 PGA scores 0-3 vs. 4 (Clear or Almost Clear or Mild or Moderate vs. Severe) and PtGA scores of 0-2 vs. 3-5 (Clear or Almost Clear or Mild vs. Moderate or Severe or Worse).

Pairwise comparisons between placebo and each active dose group was made using Fisher's exact tests at the 5% level of significance.

TABLE 1
Patient Characteristics

| Main Study | N = 66 | | N = 59 | | N = 59 | |
|---|---|---|---|---|---|---|
| Age (Mean; Years) | 45.6 | | 44.6 | | 43 | |
| Weight (Mean; kg) | 88 | | 90 | | 88 | |
| BMI (Mean; kg/m²) | 29.7 | | 30.5 | | 29.6 | |
| Baseline PASI | 17.7 | | 18.6 | | 18.1 | |
| Duration of Psoriasis | 17 | | 17 | | 16 | |
| (Mean; Years) | | | | | | |
| % Male | 61 | | 75 | | 66 | |
| PET-CT Sub-study | N = 18 | | N = 16 | | N = 13 | |
| Age (mean, range) | 50.0 | (26-68) | 48.8 | (23-59) | 48.9 | (23-59) |
| CV Risk factors: | | | | | | |
| Vascular disease | 1 | (6%) | 1 | (6%) | 0 | |
| Dyslipidemia | 6 | (33%) | 7 | (44%) | 2 | (15%) |
| Statin users | 6 | (33%) | 6 | (38%) | 1 | (8%) |
| LDL baseline | 2.62 | | 3.22 | | 2.95 | |
| (mmol/L, mean) | | | | | | |
| Diabetes | 3 | (17%) | 3 | (19%) | 1 | (8%) |
| Obesity | 15 | (83%) | 13 | (81%) | 11 | (85%) |

### Results:

Statistically significant improvement in psoriasis endpoints of Physician and patient global assessments were demonstrated in the main study, with a dose response pattern. No related Serious Adverse Events were observed. No deaths occurred on therapy. Only a low overall rate of Serious Adverse Events (1.6%, 2 subjects) were observed. A low overall rate of discontinuation due to Adverse Events was observed: 3.2% (4 subjects) on VB-201; and 3.4% (2 subjects) on Placebo. A small excess of diarrhea in the VB-201 80 mg/day (15.3%) vs placebo (6.8%) was observed. However, all cases were mild (Grade 1) and transient (≦one week in 8 of 9 VB-201 80 mg/day subjects) and no patients discontinued treatment due to diarrhea.

### Physicians Global Assessment Grouped Categorical Scores 0-3 vs. 4

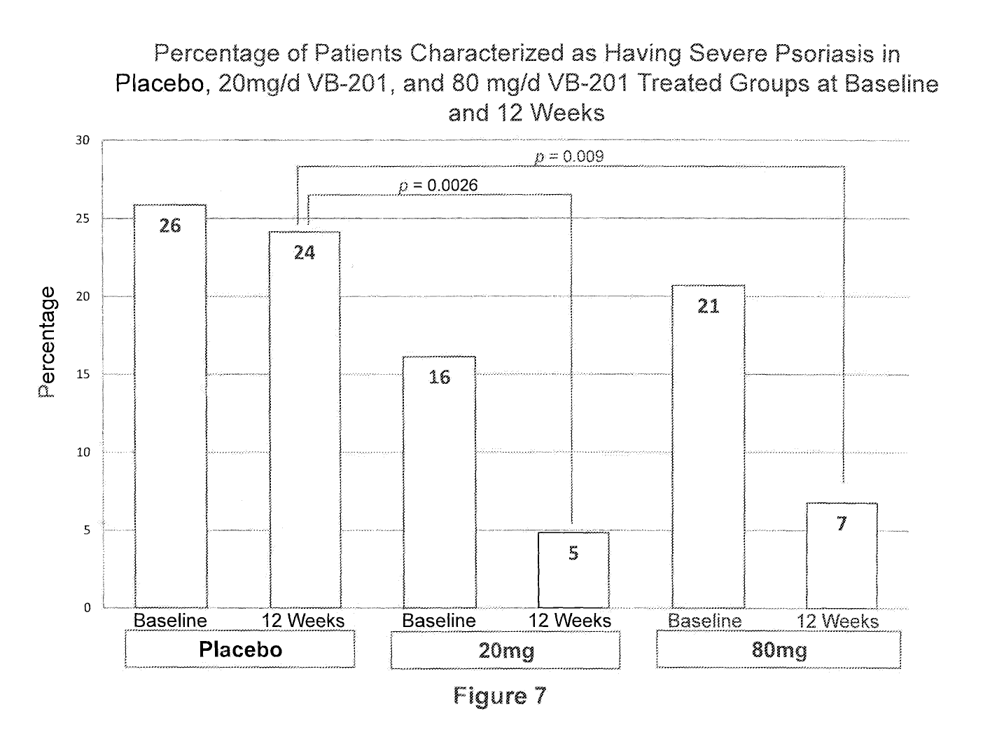

PGA frequency and percentage of grouped categorical scores 0-3 vs. 4 (Clear or Almost Clear or Mild or Moderate vs. Severe) at Baseline, Week 8 and Week 12 are presented in Table 2, below. A statistically significant difference was observed in both the 20 mg and 80 mg treatment groups at weeks 8 and 12 when compared to placebo (20 mg: week 8, p=0.002 week 12, p=0.003. 80 mg: week 8, p=0.043, week 12, p-0.011). At week 8 in the severe (4) category, there were 13 (22.4%) patients in the placebo group, 2 patients (3.2%) in the 20 mg group and 5 patients (8.5%) in the 80 mg group. At week 12 in the severe (4) category, there were 14 (24.1%) patients in the placebo group 3 patients (4.8%) in the 20 mg group and 4 patients (6.8%) in the 80 mg group. The above results are illustrated in FIG. 7.

TABLE 2
Physician Global Assessment (Grouped Categorical Response: 0-3 vs. 4)

| | | Placebo | | VB-201 20 mg/day | | VB-201 80 mg/day | |
|---|---|---|---|---|---|---|---|
| | | No/Almost | | No/Almost | | No/Almost | |
| | | Clear/Mild/ | | Clear/Mild/ | | Clear/Mild/ | |
| Categories 0-3 vs. 4 | | Mod | Severe | Mod | Severe | Mod | Severe |
| Baseline | Freq. | 43 | 15 | 52 | 10 | 46 | 12 |
| | % | 74.14 | 25.86 | 83.87 | 16.13 | 79.31 | 20.69 |
| | Pvalue | • | • | • | 0.261 | • | 0.661 |
| | (Fishers) | | | | | | |
| Week 8 | Freq. | 45 | 13 | 60 | 2 | 54 | 5 |
| | % | 77.59 | 22.41 | 96077 | 3.23 | 91.53 | 8.47 |
| | Pvalue | • | • | • | 0.002 | • | 0.043 |
| | (Fishers) | | | | | | |
| Week | Freq. | 44 | 14 | 59 | 3 | 55 | 4 |
| 12/EOT | % | 75.86 | 24.14 | 95.16 | 4.84 | 93.22 | 6.78 |
| | Pvalue | • | • | • | 0.003 | • | 0.011 |
| | (Fishers) | | | | | | |

### Patients Global Assessment—Grouped Categorical Scores 0-2 vs. 3-5

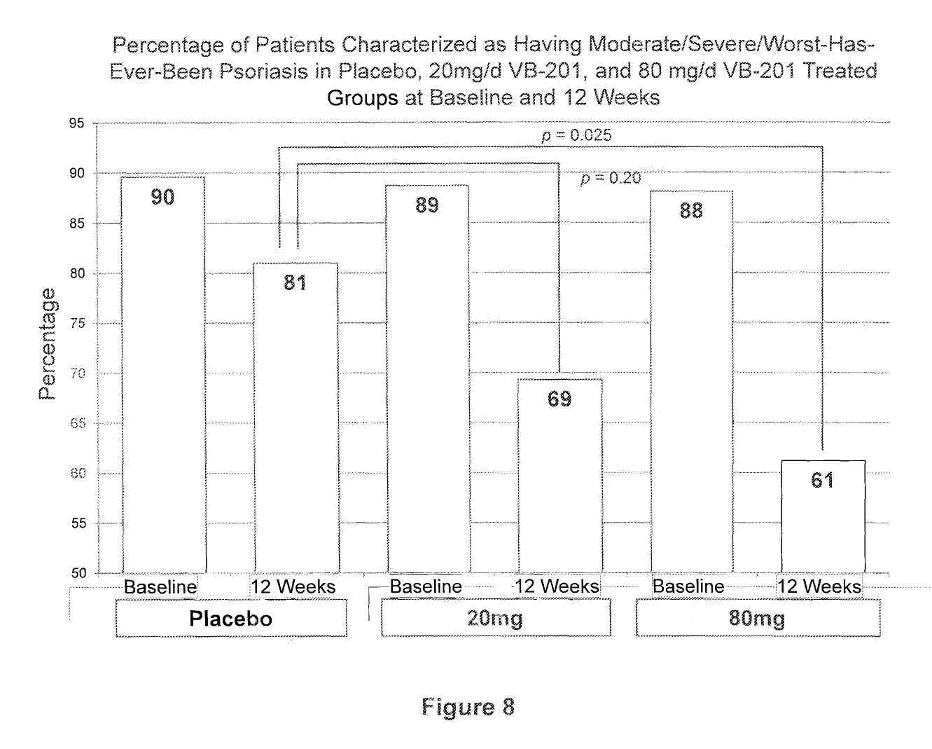

PtGA frequency and percentage of grouped categorical scores 0-2 vs. 3-5 (Clear or Almost Clear or Mild vs. Moderate or Severe or Worse) at Baseline, Week 8 and Week 12 are presented in Table 3, below. A statistically significant difference compared to placebo was observed in the 80 mg treatment groups at week 12 (p=0.025). The placebo group at week 12 had 47 patients (81.0%) in the severe or worse (3-4) categories compared to 36 patients (61.0%) in the 80 mg group. The comparison at week 8 was similar but did not quite reach statistical significance (p=0.056). The placebo group had 48 patients (82.8%) in the severe or worse (3-4) category compared to 39 patients (66.1%) in the 80 mg group. The above results are illustrated in FIG. 8.

TABLE 3
Patient Global Assessment (Grouped Categorical Response: 0-2 vs. 3-5)

| | | Placebo | | VB-201 20 mg/day | | VB-201 80 mg/day | |
|---|---|---|---|---|---|---|---|
| Categories | | No/Almost | Mod/Severs/ | No/Almost | Mod/Severs/ | No/Almost | Mod/Severs/ |
| 0-2 vs. 3-5 | | Clear/Mild | Worse | Clear/Mild | Worse | Clear/Mild | Worse |
| Baseline | Freq. | 6 | 52 | 7 | 55 | 7 | 52 |
| | % | 10.34 | 89.66 | 11.29 | 88.71 | 11.86 | 88.14 |
| | Pvalue | • | • | • | 1.000 | • | 1.000 |
| | (Fishers) | | | | | | |
| Week 8 | Freq. | 10 | 48 | 20 | 42 | 20 | 39 |
| | % | 17.24 | 82.76 | 32.26 | 67.74 | 33.90 | 66.10 |
| | Pvalue | • | • | • | 0.062 | | 0.056 |
| | (Fishers) | | | | | | |
| Week | Freq. | 11 | 47 | 19 | 43 | 23 | 36 |
| 12/EOT | % | 18.97 | 81.03 | 30.65 | 69.35 | 38.98 | 61.02 |
| | Pvalue | • | • | • | 0.205 | • | 0.025 |
| | (Fishers) | | | | | | |

### Conclusion:

VB-201 at doses of 20 mg/day and 80 mg/day is well tolerated by patients. VB-201 has an anti-inflammatory effect in chronic plaque psoriasis.

The results demonstrated proof of concept for an anti-psoriasis effect in patients with moderate to severe plaque psoriasis. While the primary efficacy endpoint of the study, change in the PASI 75 (decrease of at least 75% in the psoriasis activity score index) response rate at week 12, was not met, the study met several of the secondary endpoints including improvement in the physician and patient global assessments. Trends for favorable efficacy of VB-201 compared to placebo were observed for PASI 50 response rates and reductions in Bovine Serum Albumin (BSA).

For PGA assessments, the decrease from baseline to week 12 with VB-201 20 mg was statistically superior to that with placebo, and there was a trend for efficacy with VB-201 80 mg treatment compared to placebo. There was a statistically significant reduction in the proportion of patients rated as “severe” (grade 4 on a 0-4 scale) PGA at 12 weeks in both VB-201 20 mg (4.8%) vs placebo (24.1%, p=0.003) and in VB-201 80 mg (6.8%) vs placebo (24.1%, p=0.043) (FIG. 7).

This was further supported by ordered logistic regression under the proportional odds model, which showed a statistically significant effect was also detected when both 20 mg and 80 mg treatment groups were combined together (p-value=0.049, OR=1.91; CI=1.00-3.65).

For the Patient Global Assessment endpoint, the decrease from baseline to week 12 with VB-201 80 mg treatment was statistically superior to that with placebo (p=0.02), and there was a trend for efficacy with VB-201 20 mg treatment compared to placebo (p=0.12) (FIG. 8). This was further supported using CMH, showing that a statistically significant improvement was observed in the 80 mg group at week 8 and week 12 when compared to placebo (p=0.02 and 0.03, respectively). Ordered logistic regression under the proportional odds model also showed a statistically significant effect in the 80 mg vs. placebo comparison (p-value=0.047, OR=1.96; CI=1.01-3.80).

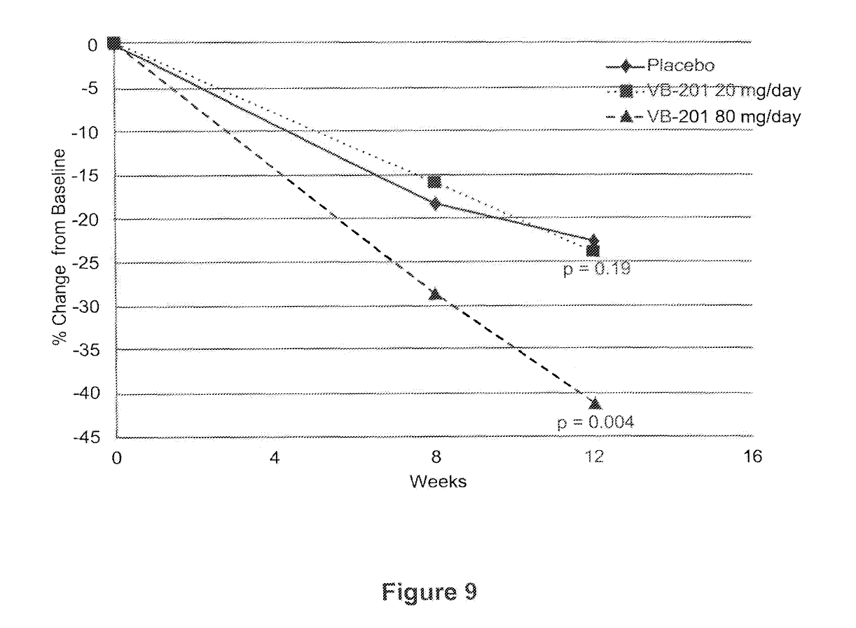

Furthermore, in a sub-population of patients, who had a baseline PAST score of about 14.3 to about 18.5, the percent change from baseline in PAST at 12 weeks were significantly dependent on the trough level of VB-201 (lowest level of VB-201 present in the patients' blood measured just before the administration of the next dose). See FIG. 9.

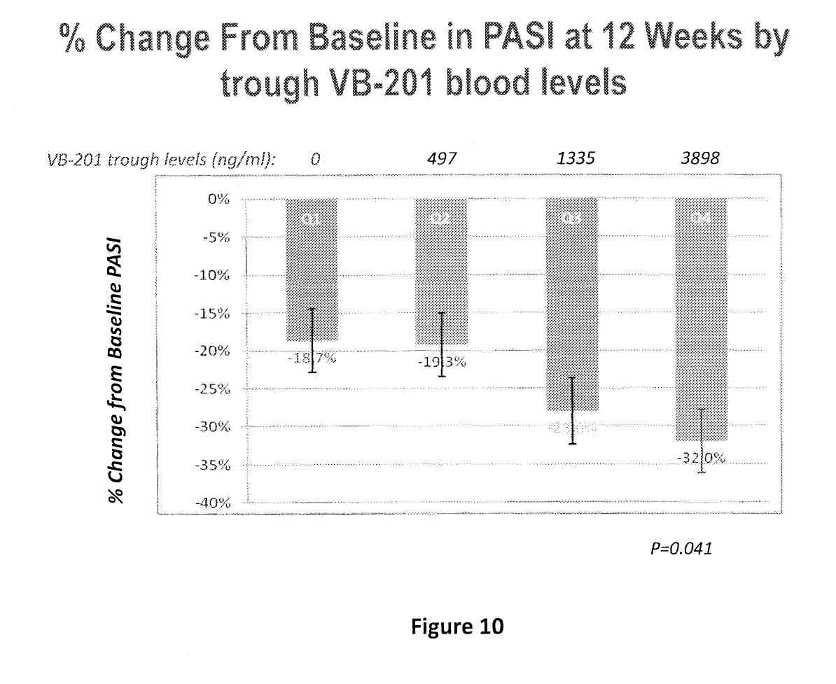

There was a statistically significant dose response as demonstrated by correlation between VB-201 plasma levels and PASI, PGA (p=0.04) and PtGA (p=0.001) efficacy endpoints. See FIG. 10. Higher plasma levels of VB-201 (higher 2 quartiles) were associated with statistically greater improvements in Total PASI score than lower plasma levels of VB-201 (the lower 2 quartiles) (p=0.04). Patients in quartile 1, who had a mean VB-201 trough level of about 0 ng/mL, experienced an average reduction of PASI score of about 18%, while patients in quartile 4, who had a mean VB-201 trough level of about 3,640 ng/mL, experienced an average reduction of PASI score of about 45% (p=0.009).

Exploratory analyses demonstrated greater trends for efficacy with the VB-201 treatment groups compared to placebo in the following subgroups of patients: baseline PASI score of 14.3-18.5 (FIG. 9), age ≦46 years, BSA 16%-24%, duration of psoriasis up to 20 years, no previous treatment with immunosuppressants or biologicals, and patients with elevated baseline high sensitivity CRP results.

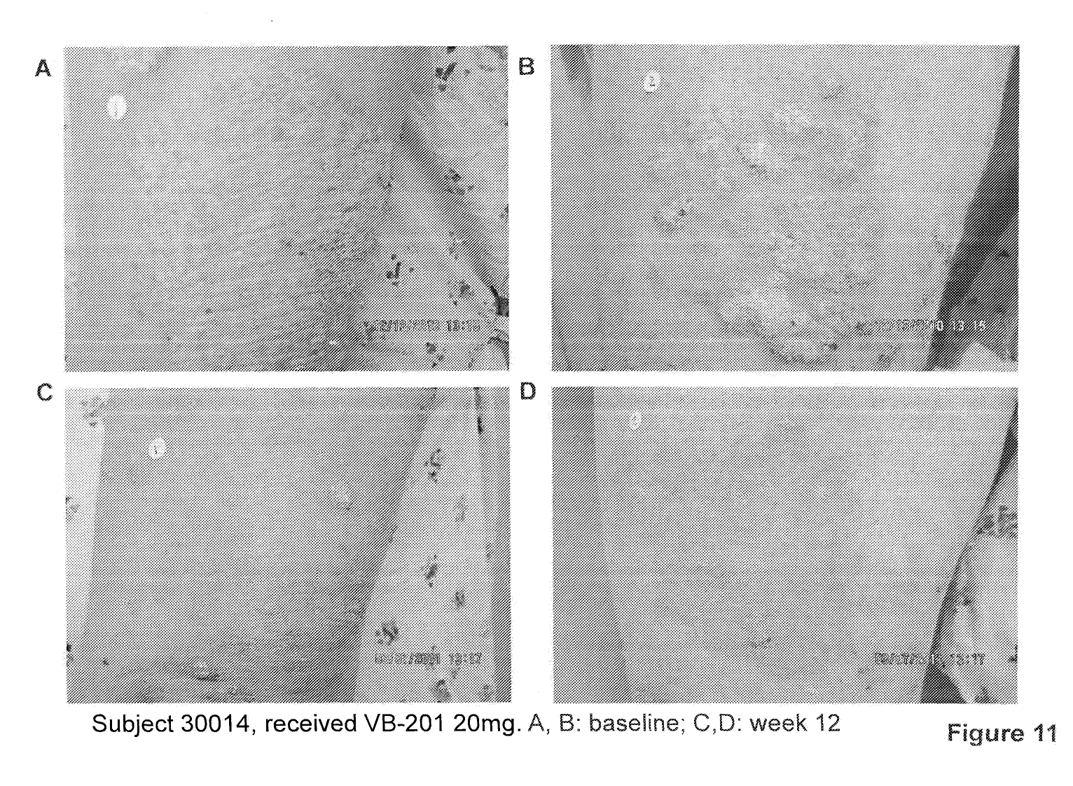

FIG. 11 further shows the images of skin lesions in a patient who received VB-201 20 mg at baseline (A,B) and after 12 weeks of treatment (C,D).

The changes from baseline in the secondary endpoints (PASI 50 response rates, BSA, PGA, and Patient Global Assessment) are suggestive of efficacy with VB-201 in psoriatic patients. Furthermore, the efficacy endpoints continued to improve overtime and had not reached a plateau at 3 months.

Incorporated herein by reference in their entirety are the disclosures of International Patent Application Publication Numbers WO2004/106486, WO 2011/083465, WO 2011/083467 and WO 2011/083469. VB-201, also named CI-201, is identified with its chemical structure and name at least in WO2004/106486 or WO2011/083465.

### Example 4

### Clinical Study to Further Evaluate VB-201 in Patients with Psoriasis

A randomized, double-blind, dose-ranging, placebo-controlled Phase II clinical study can be conducted to evaluate the efficacy and safety of orally administered VB-201 in patients with moderate to severe plaque psoriasis. For example, VB-201 may be tested in male or female patients ≧18 to ≦75 of age with moderate to severe, stable, active plaque psoriasis vulgaris affecting between 10% to 30% of the body surface and with a Psoriasis Area and Severity Index (PASI) score of 10 to 20. Such study may examine the effect of treatment with two different doses of VB-201 compared to placebo for 16 weeks on measures of disease activity in patients with psoriasis, and to examine the safety and tolerability of up to 24 weeks' treatment with VB-201 compared to placebo in patients with psoriasis. The following exemplary study design may be employed:

Study Design: Stage 1:

The initial 16 weeks of this study can be a double-blind, parallel-group, placebo-controlled randomized study with oral administration of VB-201 at doses of 80 mg/day or 160 mg/day (80 mg BID) or placebo. Subjects can be screened for eligibility and then, up to 28 days later, at the baseline visit, randomized to one of three treatment groups (1:2:2): VB-201 80 mg/day, VB-201 160 mg (80 mg BID) or placebo, respectively.

Study Design: Stage 2:

At week 16, VB-201 subjects can continue to receive blinded treatment with the same dose assigned in the initial phase; placebo subjects can cross over to VB 201 160 mg (80 mg BID) for an additional 8 weeks. Each subject can have a final safety visit 4 weeks after stopping treatment with study drug.

Dosage Regimen and Treatment Groups:

VB-201 can be administered orally: 80 mg/day (80 mg QD); 160 mg/day (80 mg BID); placebo. In order to maintain the study blind, all subjects can administer study medication in the morning (2 capsules) and in the evening (2 capsules). Subjects randomized to the VB-201 80 mg/day can receive 80 mg in the morning and placebo in the evening. Morning (AM) and Evening (PM) doses of study medication can be taken with food 12±2 hours apart.

Exemplary Number of Subjects:

Approximately 180 subjects [e.g., targeting 72 subjects in the VB-201 160 mg (80 mg BID) and placebo treatment groups and 36 subjects in the VB-201 80 mg group] can be enrolled into the placebo-controlled initial phase of this study.

Exemplary Duration of Participation:

Subjects can participate in the study for up to 32 weeks with up to 4 weeks for screening and establishment of baseline, followed by 16 weeks (Stage 1) of blinded treatment, 8 additional weeks (Stage 2) of double-blind treatment with VB-201 at a dose of either 80 mg/day or 160 mg (80 mg BID) and a follow-up visit 4 weeks after their last dose of study medication.

Exemplary Eligibility Criteria—Inclusion Criteria:

1. Fully understand all elements of and have signed and dated the written Institutional Review Board (IRB) or Independent Ethics Committee (IEC) approved informed consent before initiation of protocol-specified procedures. 2. Male or female subjects ≧18 to ≦75 years of age, who have a diagnosis of chronic plaque psoriasis for at least 6 months prior to screening. 3. Plaque psoriasis covering between 10% to 30% of body surface area (BSA). 4. PASI severity moderate to severe, scoring at least 10 but not higher than 20. 5. For a female subject; either: subject is of non-childbearing potential, defined as: menopause with amenorrhea ≧2 years, hysterectomy, or bilateral oophorectomy or—agrees to continue to use adequate contraception (implants, injectables, combined oral contraceptives, intrauterine devices, sexual abstinence or vasectomised partner) throughout the study and for at least one month following termination and have a negative pregnancy test at screening and before the first dose of study drug; males must use at least one method of contraception (e.g., condom) throughout the study. 6. In the opinion of the investigator, the subject will be compliant and have a high probability of completing the study and all required procedures.

Exemplary Exclusion Criteria:

Subjects who meet ANY of the following criteria can be excluded from participation in this study: 1. The subject presents with psoriasis that is predominantly guttate, erythrodermic, inverse, pustular or palmo-plantar or an unstable form of psoriasis. 2. Received any investigational drug within 30 days of screening. 3. Previous participation in a VB-201 study. 4. Previously received or is currently receiving systemic biologic treatment for psoriasis (e.g. ustekinumab, adalimumab, etanercept, etc). 5. The subject has not undergone wash-out periods of sufficient duration for the following treatments at Baseline: Topical psoriasis treatments: 2 weeks; Systemic (non-biologic) psoriasis treatments: 4 weeks or 5 half-lives (whichever is longer); Phototherapy: 4 weeks. 6. The subject anticipates getting enough ultra-violet light during the study (e.g. sunbathing; tanning salon, etc.) to cause psoriasis to improve. 7. The subject has a known allergy or sensitivity to the study treatment(s) or to any of the excipients contained in the study drug formulation. 8. Any other acute or chronic medical condition that, in the opinion of the investigator, increases the risk to the subject or the likelihood that the subject will be unable to complete the study. 9. Subjects with any laboratory test at screening that common medical practice would deem as significantly abnormal. The following will be deemed as significantly abnormal. 10. History of cancer, the exception is skin cancer. 11. Has a clinically significant systemic infection (e.g., chronic or acute infection, UTI, URI) within 30 days of baseline, or a history or presence of recurrent or chronic infection [e.g. viral infections, (including hepatitis B or C, HIV), bacterial infections, systemic fungal infections, or syphilis]. 12. Evidence of tuberculosis as indicated by a positive Quantiferon test at screening or within 30 days prior to screening. 13. Chest X-ray within 3 months of screening visit suggestive of tuberculosis or active fungal infection. 14. History of substance abuse, including alcohol abuse, within the past year. 15. Has a history of or has a current, clinically significant major psychiatric disorder. 16. Female subject with a positive pregnancy test or nursing, or planning a pregnancy during the course of the study. 17. Subjects with a QTc-prolongation >470 msec, risk factors for torsades de pointes such as heart failure NYHA II-IV, hypokalaemia, family history of long QT syndrome and subjects using concomitant medication that prolongs the QT interval. 18. Unwilling or unable to comply with study requirements.

Exemplary Concomitant Medications:

Use of emollients on any lesions and tar-based shampoos can be permitted except on days of study visits. Topical corticosteroids, including those with low-potency, may not be permitted; their use can be precluded for at least 2 weeks prior to the baseline visit. Other treatments for psoriasis (including other topical and systemic psoriasis treatments, phototherapy and biologic treatments) may be prohibited as mentioned in the eligibility criteria. In addition, previous use of a biologic treatment may exclude subjects from study entry.

Exemplary Safety Endpoints:

Safety can be assessed based on all subjects in the study who have received at least one dose of study drug (Safety Population): Physical exam; adverse events; vital signs; laboratory values—clinical chemistry, hematology, urinalysis; ECGs.

Exemplary Stage 1 Primary Efficacy Endpoint:

The proportion of subjects in the VB-201 160 mg (80 mg BID) treatment group who achieve at least 50% improvement from the baseline PASI score at Week 16 (PASI 50) compared to the proportion of PASI 50 responders in the placebo group.

Stage 1 Secondary Endpoints:

1. Proportion of subjects in each of the VB-201 treatment groups and in the combined (both dose groups) VB-201 treatment groups who achieve at least 75% improvement from the baseline PASI score (PASI 75) at week 16 compared to the proportion of PASI 75 responders in the placebo group. 2. The mean change in the PASI score from baseline to week 16 in each of the two VB-201 treatment groups and in the combined (both dose groups) VB-201 treatment groups compared to the mean change in the placebo group. 3. Change in affected Body Surface Area (BSA) from baseline to week 16 in each of the VB-201 treatment groups and in the combined (both dose groups) VB-201 treatment groups compared to placebo. 4. Change in PGA scores from baseline to week 16 in each of the VB-201 treatment groups and in the combined (both dose groups) VB-201 treatment groups compared to the placebo group, to include analyses of: (a) proportion with PGA score of 0-1, and (b) proportion with PGA score of 4-5. 5. Change in Patient Psoriasis Global Assessment scores from baseline to week 16 in each of the VB-201 treatment groups and in the combined (both dose groups) VB-201 treatment groups compared to the placebo group. 6. The proportion of subjects in the VB-201 80 mg/day treatment group who achieve at least 50% improvement from the baseline PASI score at Week 16 (PASI 50) compared to the proportion of PASI 50 responders in the placebo group.

Exemplary Stage 1 Tertiary Endpoints:

1. Change in itching VAS (only in subjects having itching at baseline rated at 10 mm on a 100 mm VAS scale) from baseline to week 16 in each of the VB 201 treatment groups and in the combined (both dose groups) VB-201 treatment groups compared to placebo. 2. Change in pain VAS (only in subjects with pain at baseline rated at 10 mm on a 100 mm VAS scale) from baseline to week 16 in each of the VB 201 treatment groups and in the combined (both dose groups) VB-201 treatment groups compared to placebo. 3. Change in the product of PGA×BSA from baseline to week 16 in each of the VB 201 treatment groups and in the combined (both dose groups) VB-201 treatment groups compared to placebo. 4. Change in the DLQI scores from baseline to week 16 in each of the VB 201 treatment groups and in the combined (both dose groups) VB-201 treatment groups compared to placebo.

Exemplary Stage 2 Primary Efficacy Endpoint:

The proportion of subjects in the VB-201 160 mg (80 mg BID) treatment group who achieve at least 50% improvement from the baseline PASI score at Week 24 (PASI 50) compared to the proportion of PASI 50 responders in the placebo group.

Exemplary Stage 2 Secondary Endpoints:

1. Proportion of subjects in each of the VB-201 treatment groups and in the combined (both dose groups) VB-201 treatment groups who achieve at least 75% improvement from the baseline PASI score (PASI 75) at week 24 compared to the proportion of PASI 75 responders in the placebo group. 2. The mean change in the PASI score from baseline to week 24 in each of the two VB-201 treatment groups and in the combined (both dose groups) VB-201 treatment groups compared to the mean change in the placebo group. 3. Change in affected Body Surface Area (BSA) from baseline to week 24 in each of the VB-201 treatment groups and in the combined (both dose groups) VB-201 treatment groups compared to placebo. 4. Change in PGA scores from baseline to Week 24 in each of the VB-201 treatment groups and in the combined (both dose groups) VB-201 treatment groups compared to the placebo group, to include analyses of: (a) proportion with PGA score of 0-1, and (b) proportion with PGA score of 4-5. 5. Change in Patient Psoriasis Global Assessment scores from baseline to Week 24 in each of the VB-201 treatment groups and in the combined (both dose groups) VB-201 treatment groups compared to the placebo group.

Exemplary Stage 2 Tertiary Endpoints:

1. Change in itching VAS (only in subjects having itching at baseline rated at 10 mm on a 100 mm VAS scale) from baseline to week 24 in each of the VB 201 treatment groups and in the combined (both dose groups) VB-201 treatment groups compared to placebo. 2. Change in pain VAS (only in subjects with pain at baseline rated at 10 mm on a 100 mm VAS scale) from baseline to week 24 in each of the VB 201 treatment groups and in the combined (both dose groups) VB-201 treatment groups compared to placebo. 3. Change in the product of PGA×BSA from baseline to week 24 in each of the VB 201 treatment groups and in the combined (both dose groups) VB-201 treatment groups compared to placebo. 4. Change in the DLQI scores from baseline to week 24 in each of the VB 201 treatment groups and in the combined (both dose groups) VB-201 treatment groups compared to placebo. Note: For week 24 placebo group efficacy responses, modeling of the trajectory of response for the placebo group during weeks 0-16 is to be used to determine the placebo week 24 values for all endpoints.

Compliance Measures:

Standard counts of unused medication and/or trough plasma levels of VB-201 can be assessed.

Exemplary Study Conduct:

Patients with stable active plaque psoriasis who meet the study eligibility criteria can be identified and considered for screening. Washout from current medications can be allowed after the subject signs an informed consent. Following screening, eligible subjects can be randomized to one of the treatment arms using a pre-established randomization list. Subjects can visit the clinic at screening and baseline and after 2, 4, 8, 12, 16, 20 and 24 weeks of treatment. In addition, all subjects (including those who withdraw prematurely) can return for a final follow-up visit at 4 weeks after their last dose of study medication. Data collection can be via electronic data capture (EDC) system CRFs.

Exemplary Statistical Methods:

The primary efficacy analyses can be completed in the Modified Intent-To-Treat (MITT) population. This population can include all subjects randomized who received at least one dose of study medication and had at least one efficacy evaluation (i.e. PASI score) after study treatment was begun. Efficacy analyses can also be completed in a Per-Protocol population; these can be exploratory and will be defined in the Statistical Analysis Plan (SAP). Categorical data can be presented as counts and percentages. Continuous data can be presented as summary statistics. All statistical comparisons can be two-sided at the 5% level of significance.

What is claimed is:

1. A method of treating vascular inflammation in a subject suffering from a chronic autoimmune or chronic inflammatory disease, the method comprising administering to the subject a therapeutically effective amount of VB-201.

2. A method of decreasing vascular inflammation in a subject suffering from a chronic autoimmune or inflammatory disease when compared to the vascular inflammation prior to the administering (base line), the method comprising administering to the subject a therapeutically effective amount of VB-201.

3. The method of claim 1 or 2, wherein the therapeutically effective amount is administered to the subject for at least about 8 weeks, at least about 12 weeks, at least about 16 weeks, at least about 18 weeks, at least about 20 weeks, or at least about 24 weeks.

4. The method of claim 3, wherein the vascular inflammation is reduced by at least about 5%, at least about 6%, at least about 8%, at least about 10%, at least about 12%, or at least about 14%, when compared to the vascular inflammation prior to the administering (base line).

5. The method of any one of claims 1 to 4, wherein the therapeutically effective amount is from about 20 mg/day to about 160 mg/day.

6. The method of claim 5, wherein the therapeutically effective amount is from about 80 mg/day to about 160 mg/day.

7. The method of claim 5, wherein the therapeutically effective amount is about 20 mg/day, about 40 mg/day, about 60 mg/day, about 80 mg/day, about 100 mg/day, about 120 mg/day, about 140 mg/day, or about 160 mg/day.

8. The method of claim 5, wherein the therapeutically effective amount is about 80 mg/day, 120 mg/day, or about 160 mg/day.

9. The method of claim 5, wherein the therapeutically effective amount is about 80 mg/day administered to the subject in 1 or 2 daily doses.

10. The method of claim 5, wherein the therapeutically effective amount is about 120 mg/day to about 160 mg/day administered to the subject in 2 daily doses.

11. The method of claim 10, wherein the therapeutically effective amount is about 160 mg/day administered to the subject in 2 daily doses.

12. The method according to any one of claims 1 to 11, wherein the chronic autoimmune or inflammatory disease is psoriasis.

13. The method of claim 12, wherein the vascular inflammation is associated with a cardiovascular disease, a peripheral vascular disease, a coronary artery disease, a cerebral vascular disease, a renal artery stenosis, an ischemic disease, or an aortic aneurism.

14. The method of claim 12, wherein the vascular inflammation is associated with an ischemic heart disease, atherosclerosis, acute coronary syndrome, unstable angina, stable angina, or stroke.

15. A method of treating vascular inflammation, the method comprising administering to the subject in need thereof a therapeutically effective amount of VB-201, wherein the therapeutically effective amount is from about 120 mg/day to about 160 mg/day administered to the subject in two daily doses.

16. The method of claim 15, wherein the therapeutically effective amount is about 160 mg/day administered to the subject in 2 daily doses.

17. The method according to any one of claims 1 to 16, wherein the subject suffers from atherosclerosis.

18. The method according to any one of claims 1 to 17, wherein the vascular inflammation is inflammation of a carotid artery.

19. The method according to any one of claims 1 to 17, wherein the vascular inflammation is inflammation of an aorta.

20. The method of any one of claims 1 to 19, wherein the vascular inflammation is measured using positron emission computed tomography (PET/CT) imaging quantifying 18-fluorodeoxyglucose (18-FDG) uptake as a target to background ratio (TBR).

21. A method of treating severe psoriasis (psoriasis of category 4 according to the Physician Global Assessment (PGA) scale), the method comprising administering to a subject in need thereof a therapeutically effective amount of VB-201, wherein the therapeutically effective amount is from about 20 mg/day to about 160 mg/day, from about 20 mg/day to about 80 mg/day, or from about 80 mg/day to about 160 mg/day for a treatment period of at least about 8 weeks, at least about 12 weeks, at least about 16 weeks, or at least about 24 weeks.

22. The method of claim 21, wherein the severe psoriasis improves to moderate, mild, almost clear or no psoriasis (psoriasis of categories 0-3 according to PGA scale) during the treatment period.

23. A method of treating moderate, severe, or worst psoriasis has ever been (psoriasis of categories 3-5 according to the Patient Global Assessment (PtGA) scale), the method comprising administering to a subject in need thereof a therapeutically effective amount of VB-201, wherein the therapeutically effective amount is from about 20 mg/day to about 160 mg/day, from about 20 mg/day to about 80 mg/day, or from about 80 mg/day to about 160 mg/day for a treatment period of at least about 8 weeks, at least about 12 weeks, at least about 16 weeks, or at least about 24 weeks.

24. The method of claim 23, wherein the moderate, severe, or worst psoriasis has ever been improves to mild, almost clear or no psoriasis (psoriasis categories 0-2 according to PtGA scale) during the treatment period.

25. A method of treating psoriasis comprising administering to a subject in need thereof a therapeutically effective amount of VB-201, wherein the therapeutically effective amount is from about 120 mg/day to about 160 mg/day administered to the subject in 2 daily doses.

26. The method of any one of claims 21 to 25, wherein the subject, prior to the administering, has a PASI score from about 10 to about 20, or from about 14 to about 19.

27. A method of treating psoriasis, the method comprising administering to a subject in need thereof a therapeutically effective amount of VB-201, wherein the therapeutically effective amount is 160 mg/day administered in 2 daily doses, wherein the subject prior to the administering the VB-201 has a PASI score that is from about 10 to about 20, or from about 14 to about 19.

28. The method of any one of claims 21 to 27, wherein the subject, prior to the administering, has psoriasis characterized by a body surface area (BSA) from about 20% to about 30%, or from about 16% to about 24%.

29. A method of treating psoriasis, the method comprising administering to a subject in need thereof a therapeutically effective amount of VB-201, wherein the therapeutically effective amount is 160 mg/day administered in 2 daily doses.

30. The method of claim 29, wherein the subject prior to the administering the VB-201 has psoriasis characterized by a body surface area (BSA) from about 10% to about 30%, or about 15% to about 25%, or about 16% to about 24%.

31. The method of claim 29, wherein the subject, prior to the administering, has a PASI score that is from about 10 to about 20, or from about 14 to about 19.

32. The method of any one of claims 21 to 31, wherein the subject has not been treated with a biologic psoriasis treatment or an immunosuppressant prior to the administering.

33. A method of treating moderate to severe psoriasis in a subject in need thereof, the method comprising administering to the subject a therapeutically effective amount of VB-201 for a treatment period.

34. The method of claim 33, wherein: a) the subject has a PASI score of 10 to 20 prior to the treatment period; b) the subject has a BSA of 10% to 30% prior to the treatment period; c) the subject was not treated with an anti-psoriatic biologic or an immunosuppressant drug prior to the treatment period; or any combination thereof.

35. The method of claim 33 or 34, wherein the therapeutically effective amount of VB-201 is 20 mg/day to 240 mg/day.

36. The method of any of claims 33 to 35, wherein the therapeutically effective amount of VB-201 is 80 mg/day to 160 mg/day.

37. The method of any of claims 33 to 36, wherein the therapeutically effective amount of VB-201 is 160 mg/day.

38. The method of any of claims 33 to 37, wherein the therapeutically effective amount of VB-201 is administered in two daily sub-doses.

39. The method of any of claims 33 to 38, wherein the therapeutically effective amount of VB-201 is administered in two daily sub-doses of 80 mg.

40. The method of claim 37 or claim 39, wherein the two daily sub-doses are administered 10 to 14 hours apart.

41. The method of any of claims 33 to 36, wherein the therapeutically effective amount of VB-201 is 80 mg/day, wherein the treatment period is at least 16 weeks or at least 24 weeks.

42. The method of any of claims 33 to 40, wherein the therapeutically effective amount of VB-201 is administered for a treatment period of at least 8 weeks, at least 12 weeks, at least 16 weeks, or at least 24 weeks.

43. The method of claim 42, wherein the treatment period is at least 24 weeks.

44. The method of any of claims 33-43, wherein the subject prior to the treatment period has a PASI score of 10 to 20.

45. The method of claim 44, wherein the subject prior to the treatment period has a PASI score of 14 to 20.

46. The method of claim 44, wherein the subject prior to the treatment period has a PASI score of 14.3 to 18.5.

47. The method of any of claims 33-46, wherein the subject prior to the treatment period has a body surface area (BSA) from 10% to 30%.

48. The method of any of claims 33-47, wherein the subject prior to the treatment period has BSA from 14 to 26%.

49. The method of any of claims 33-48, wherein the subject was not treated with a biologic psoriasis treatment or an immunosuppressant prior to the treatment period.

50. The method of any of claims 33-49, wherein the subject has a diagnosis of chronic plaque psoriasis for at least 6 months prior to administering the VB-201.

51. The method of any of claims 33-50, wherein the subject undergoes a concurrent phototherapy.

52. The method of any of claims 33-51, further comprising administering to the subject a therapeutically effective amount of an additional therapeutic agent for psoriasis.

53. The method of claim 52, wherein the additional therapeutic agent for psoriasis is a topical agent for psoriasis.

54. The method of any of claims 33-53, wherein the psoriasis is plaque psoriasis.

55. A method of treating or reducing inflammation associated with an implant in a subject in need thereof, the method comprising administering to the subject a therapeutically effective amount of VB-201.

56. The method of claim 55, wherein the inflammation associated with an implant is a local inflammation or a systemic inflammatory reaction.

57. The method of claim 55 or 56, wherein the implant is a silicone, a saline, a metal, a plastic, or a polymeric implant.

58. The method of claim 55 or 56, wherein the implant is a cosmetic implant, a prosthetic implant, a subdermal implant, a transdermal implant, a bone replacement implant, or a bone fracture repair device.

59. The method of claim 55 or 56, wherein the implant is a drug delivery implant or a drug release implant.

60. The method of claim 55 or 56, wherein the implant is selected from the group consisting of an artificial joint, an artificial heart, an artificial heart valve, a testicular prosthesis, a breast implant, a dental implant, an ocular implant, a cochlear implant, a penile implant, a cardiac implant, a catheter, an implantable urinary continence device, a pacemaker, an electrode, a Hernia support device, or a respirator tube.

61. The method of any of claims 55 to 60, wherein the implant is a breast implant.

62. The method of any of claims 55 to 61, wherein the therapeutically effective amount of VB-201 is 20 mg/day to 240 mg/day.

63. The method of any of claims 55 to 62, wherein the therapeutically effective amount of VB-201 is 80 mg/day, 120 mg/day, or 160 mg/day.

64. The method of any of claims 55 to 63, wherein the therapeutically effective amount of VB-201 is administered in two daily sub-doses.

65. The method of any of claims 55 to 64, wherein the therapeutically effective amount of VB-201 is administered for a treatment period of at least 8 weeks, at least 12 weeks, at least 16 weeks, or at least 24 weeks.

66. The method of any of claims 1 to 65, wherein the therapeutically effective amount of VB-201 is administered orally.

67. The method of any of claims 1 to 66, further comprising administering to the subject in need a therapeutically effective amount of an additional therapeutic agent.

68. The method of any one of the preceding claims, wherein the therapeutically effective amount of VB-201 is formulated in a pharmaceutical composition, wherein the pharmaceutical composition comprises a thermosoftening carrier.

69. The method of claim 68, wherein the thermosoftening carrier is selected from the group consisting of waxes, poloxamers, macrogol glycerides, high-molecular weight PEGs, glycerol monooleates or monostearates, hydrogenated or partially hydrogenated glycerides, Gelucires, and hard fats.

70. The method of claim 69, wherein the thermosoftening carrier is selected from the group consisting of PEG6000, poloxamer 188, and combinations thereof.

71. The method of claim 70, wherein the thermosoftening carrier is poloxamer 188.

72. The method of any of claims 68-71, wherein the pharmaceutical composition further comprises an anti-adherent agent.

73. The method of claim 72, wherein the anti-adherent agent in the pharmaceutical composition is selected from the group consisting of talc, magnesium stearate, cellulose, cellulose derivatives, lactose, gelatin, alginates, aluminium hydroxide, magnesium oxide, clays, attapulgite, bentonite, carrageenan, copovidone, hectorite, polymethacrylates, sodium docusate, erythritol, povidones, croscarmellose sodium, dextrates, starches, iron oxide, kaolin, silicates, corn flour, sugars, calcium carbonate, magnesium carbonate, calcium phosphate, calcium sulfate, bicarbonates, citrate salts, and titanium dioxide.

74. The method of claim 72, wherein the anti-adherent agent in the pharmaceutical composition is talc.

75. The method of any of claims 72-74, wherein the pharmaceutical composition has a weight ratio of the anti-adherent agent to VB-201 of 1:5 to 5:1.

76. The method of any of claims 72-75, wherein the pharmaceutical composition has a weight ratio of the anti-adherent agent to VB-201 of 1:4 to 1:1.

77. The method of any of claims 72-76, wherein the pharmaceutical composition has a weight ratio of the anti-adherent agent to VB-201 of 1:1.

78. The method of any of claims 72-75, wherein the pharmaceutical composition has a weight ratio of the anti-adherent agent to VB-201 of 1:4.

79. The method of any of claims 72-78, wherein the pharmaceutical composition has a concentration of the anti-adherent agent of 1% to 45% by weight of total weight of the pharmaceutical composition.

80. The method of any of claims 68-79, wherein the pharmaceutical composition further comprises a thixotropic agent, a gelling agent, or a combination thereof.

81. The method of any of claims 68-80, wherein the pharmaceutical composition comprises a thixotropic agent.

82. The method of claim 81, wherein the pharmaceutical composition comprises a thixotropic agent selected from the group consisting of fumed silica, kieselguhr, gums, cellulose derivatives, starches, polymers, emulsifiers, and clay derivatives, attapulgite, mica, synthetic magnesium phyllosilicates, layered silicates, modified smectites, hectorite, and sepiolite.

83. The method of claim 81, wherein the thixotropic agent in the pharmaceutical composition is fumed silica.

84. The method of any of claims 81-83, wherein the pharmaceutical composition has a concentration of the thixotropic agent of 0.25% to 10% by weight of total weight of the pharmaceutical composition.

85. The method of any of claims 68-83, wherein the pharmaceutical composition is a liquid-fill composition.

86. The method of any one of the preceding claims, wherein the subject is a human patient.
